SEQUENCE LISTING (11 sequences)

SEQ ID NO: 1 - DNA, 2560 bp, Bifidobacterium longum
aatacgactggcgcggacttcagcttcggtaatcggagcctgcgctgcggcggtgatggacgatgcgccctttgcggccggttcattggcgagcgccggggtgccgatcagggcagccggaaggaccgcagcgaacgccagagtatggaggttgaaggtcttcatcatcgggttcctttcggatcatcgtcgcgagggccgatccgcaaggcgagatattcagccctgcggttggtccgtatccgtgaaatagcccgacgccggtccgcccaaatccgccgcatggcgtgccttccggcctgcgccggatggcctatgcgtttttcggaggggatggtatctggacggggtgcacaatcgttcctcacccttgccgatccagatcggcaccgttgcggagatgcgcgatctctatcgcgccgccgaagcgcgcgcggcacggatgcgcctgctcaccgtgagtgggcaggaactggccgacgccgaccccaccaacattgccgagcgtctggcccgatctgccgaccgcctcgccttttttctcggcagccgcgatgcccaggtgatcgagggcgatggcgcagagggcattgccattgtcgcccccggtccggcgcgcaaacccgtggcacgattccgcgtcgacggaatcgcaagcctcgacgcgatggaggatatcgaagaccgggacgcagtggcgatgcatctcgaactgctgggcgcaaacccatcgaccgcatccagcgcgaagcagtgaacgtggcgccctactggctaccctgctgggagcagcgagcagcaggcgtcgagcacatggtcggccggatgttctcggcacaggaagaggagcgccgtcgggtctcgcaggagctgcacgacggggttgcacagaccgccacggcattggcgcggattctggaaggcgtcggcgaaggccaaaccgaagcgcttcccgctgcggagcgggatcggctcgcggggatcgcgcgcgcgctcgtgcgcgaactgcgcgcggtcatcggcggcttgcgcccgaccctgctggacgatctcggtctgcaggctgcgctacgttctctggcggacggccttgaagaggacgggtatcaggtttccttctgcatggccgacgatgcgtcgcggctgtctccgaccgtcgagatcgccctgttccgggttgctcaggaagcgattgccaatgtccgcaagcatgccggcggtccctgtgccgtcgccatcgcgcttcggatcgagaccgggcacctgcgcttgcagatccagcgacagcggccgcgggccgagcgatcagcctggacgctggcgcaggagccaggccgaccggtcgccatgtcggcatcgacgtcatgcatgaacgaatgagcgcaatcggcgggcacctcgaatggtcggcaggcgccgacggtggcgtgaccgttaccgcgcatctgccggaatccgcctgaccgtggccggtccgcgaatcctgatcgtcgacgaccaccagttggcgcgtgaggggctgcgcgccgtgctggcccagagcggtgtcaacgtagtcggcgtcgcgtccagcggggaggaggcgatcgatcaggtgcgtttgcttcatcccgacgtcgtcctgatggatgtgcgtcttggcagcgggatcgacgggctggaagcgacccgccggatcgccgcgctcgacaccgcgacgcggatcctgatgctcaccttgcacgacatgccggcctatgtgcgcgaggcgctcgcggcgggtgcggccggctatgtgctcaaggacaccgcgatcggcgacctgatcgccgcgatcgatcaggtgatggcgggcaacctcggccggtcccactggccctggtcaatgccgcgatgcgggcgcctgcgctaccgcagcgcgatgccgacatctcgcgcgtgctcacgtcgcgcgaacaggaggtggtggcattggtggcccgcgggcttcaccaacaaggagattgcgcgggagctggcgatccaagcccggcgacggtcaaggcgcatgtcgagcgtgtcatcggcaagcttggcgtcgccgaccgcacacaagccgccgtgctggcggcgcagatgcgcccggcagggctgtagagcgcaatggcaccagccccggccaagccgttattgcgcttctggagcaaccgtccgctcgcgctgaaggggctggtggtggtcgcgctgccgctggccatcctgctcgttgcgctggtttcgctctatcttgccagcaacgccgaaaagcgcgccgaggacgatgtccgccgcgcgttcgccattcagcgcgacacctatcaggttcacgccctgctggccgaagcggcggccggcgtacgcggctttgcgctgacgcgagaggaacgcttcctggcaccgtaccgcaaggcggaggcggagattccggtcacgatggagcggctcgatgcggcgatccgcgacccggatcgtgcggcgcgatttccagaatctcagcgaccttaccgcgcgcaaacgcgacggcctgcggcagatcgtcgcactgaccggtccggcgcgcacc

SEQ ID NO: 2 - DNA, 7001 bp, Bifidobacterium longum
gccgtcaaggccccgggctttggcgaccgtcgcaaggccatgctgcaggatatggccatcctgaccggtgctcaggttgtctccgacgaactgggcctgaagctcgagtccgtcgacacctccgtgctcggccacgccaagaaggtcatcgtctccaaggacgagaccaccatcgttcagggcgctggctccaaggaagacatcgacgctcgcgtggcccagatccgcgctgagatcgagaacaccgactccgattacgaccgtgagaagctgcaggagcgtctggccaagctggctggcggcgtggctgtcatcaaggtcggcgctgccaccgaggtcgaggccaaggagcgcaagcaccgcatcgaagatgccgtgcgtaacgccaaggccgccatcgaggaaggcctgctgcctggcggtggcgtggccctcgttcaggctgctgccaaggccgagaagaccgaggccgtcacctccctgaccggcgaagaggctaccggtgccgccatcgtgttccgcgccatcgaggccccgatcaagcagatcgccgagaacgccggcgtgtccggtgacgtggtcatcaacaccgtccgctccctgcctgatggcgaaggcttcaacgccgccaccgacacctacgaagacctgctggccgccggtgtgaccgacccggtcaaggtgacccgctccgctctgcagaacgccgcctccatcgctggtctgttcctgaccaccgaggccgtcgttgccaacaagccggagccgaagtctgctgccccggccgccggtgccgacatgggctactgatccgcaaacgatcgctagctgattgagctgaaaggggactccttcgggagtcccttttttgtattctggctcccctctttgaggggagccaagaaactagctggcaaacaggcgcgaagcttggatttcggcgtcagcgtacacggtggaggcttgcgtcagcgagcgttggatggactcgagtgaggcctccatctgctgttgggccgcacgccactgttcggctaccgcggtgaactgggtggccgccgaacctcgccatgcgtcctgcaaggcattgagattggtgtacatgccgcccacggcctgcctgatttgcgagatcgaagtggccactgcggccgaggatgattggattcgctcggaatctacctgatattggggcatcgttgctccttttctagagggttaatggtatgtatggcggccgattgccaataccggccgttaaggtggaggatatgtcaacaacgaatcgggaagacggaaaaacaactattgtggtcctagcttcggagccgtcctattcggccttcgggaatatggtccgaaaatggcttgtttgcgccatttgcgcccttgctgcagtagcgatgaacgcaatgtggataactgcaaatgcctatggggataactcgagcgacagctctgacagctccagcagcagcaccgacagcggcgtgaccatcaccgagaacatcacggatacggaaaatctcctgggctcccatgcggccgaagtcacggatgccatcgccaaaaccgagaaggaaaccggtgtacacgtgcatctgctgtatctttccagtttcaatagtcaacagaaaccgggagactgggcggcaaccgtgatggagtccatgaatcccaagccgaacacagtgatgcttgccgtggcctcgaatgacggcaacctggtggtcgtcgtgtccaagaactccgataagtggcttctcgatmacaaaaccgtcgataagctatccgaagccgcacagcagccgttactggaaaacccgccgagctggtctggcgcggcaacggcaatgatggatcaaatcgtgaaatccaaaaaagcctccacctcatcgtccacggtgatcgtaggcataatcatcatgggtgtggtattggtggcgctggtcatcatcatcgtggtcatggtggtgattcatcggcgcaaggagatcaagaaggattccaaggccgaactccaagatgacattcaggaaacgcccagaagagcgcgacactctaggaagcatgagtaagcctattgaagcatcgattgttgttgtcgacgacgagccgtccatccgagaactgctggtcgcttccctgcacttcgccggattcgaggtaaacaccgccgcttccggttctgaagccattgaggttatcgaaaaagtgcagcctgacctcatcgtgttggacgtcatgctgcctgacatcgatggattcaccgtcacccgtcgcatccgccaggaaggcattaacgcccctgtgctgtttctcaccgctcgtgacgacacccaagacaagatcatgggcctgaccgtcggcggtgacgattacgtcaccaagccgttcagcctcgaggaagtcgtggctmratcatccgcgccattctgcgccgtacccgcgaacagagtggaagacgatccgaktattcggcagtcagccggcactttggaaatcaacgaggactcccacgacgtgacccgtgcagggcagccggttgacctgagccctaccgaatacaagctgctgcgctacctgatggacaacgagggccgagtactgtccaaggcacagattctcgaccatgtctggcaatacgactggggcggcgacgcagccatccgtcgaatcagtacatctcctacctgcgcaagaaagtcgacggcatcgaggtcgacgacggcgaaggcggcaagcgcaaggtgactccgctgatcgaaaccaagcgcggcatcggatacatgattcgcgaaccgaagaactaatccgcctccatccataacgtatcgcaatctcatgagtatccccgataagcagtcaggcgcgcgtaccgccgagcagcacgccgaacgccgccccgaacacgaatcccatgcctgcaatgtcgccgttcgccgcccatttccaagagcagccgcgagctgtaagaaaaacatgctgatgcggcatatcgaccgtatctcgttgagcagcaagctggtggcctgcaccatcgccgtgctgctcatcggcgtgtcggtgatttccttctccatccgcgcgttggttgaacaactacatgctgcagaaaaccgacacccaagttaagctcgcaaagccaactggtggtcaacaatatcgatctgctgtcaaaaaatgactcatcggggccgaactcgtactttctgcagattcaatacaccgacggaaccaaagacaaagaaggcaatcctctggtcgtgaccccgctgatgccgcagatgcaggatgccgcagatgcaggacggtatagtaccggtcccgattctgcctacctatggcgataccaatggcatcacgctcggtcaggcattcactacgcaggcggtggccaagcagatcatcacggtgcagtccgacagtgcggacagccagaacgatccggccaacggaaactccaattcctctgacaccatcaccaaggtactggccaaccccactggccaatgccaaccatgccgccatcgtcacgggcgagggcaccatggcgtattctgccggtgaccttccagcaaaacggcaaggaccgtgccgtggtgtacatcggcttgtcgctggccgaccaaatcgacaccgtcaacacactcacccgatactgcattgtggtcggcatcgccgtggtgctgctcggcggctccctgtccacgctgatcatccagcacacgatgacgccgctgaagcgcatcgagaagaccgccgcgaaaattgcagccggcgacctgagccaacgtattccttccgcgccggaaaacaccgaggtcggctctctggccgcctcgctgaattccatgctcacccgaatagaatccagtttccatgagcaggaggagaccaccgacaagatgaaacggttcgtctccgacgccagccatgagctgcgcaccccgttggccgccatccatggctatgcggagctgtataagatgcagcgcgatatgcctggtgccttggagcgtgcggacgagtccatcgaacatatcgaacggtccagccagcgcatgactgttcttgttgaggatctgctgtctttggcccgtctcgatgaggggcgcggcatcgatatgaccggcacggtgaaactctcgtcgctggtcaccgacgccgtcgacgatttgcatgcgctcgacccggaccgtgccgttcgccgcatgcagatttccctcgaaccggcgcgcgatctgaaccatcccgccgaattcagccttgccgaaggcgattggcctgaggtcgtactgcccggtgatgcctctcgactgcgccaggtggtgaccaacatcgtgggcaacatccaccgctacacgcctgccgattcgcccgctgaagccgcgctcggtgtgatgccggccgccatcgatccaagacagctcgcccgcatgcccgccagcgacgcgtcaatgcggcggttcatcgacgctgccgaagtaggtgcctcgatgcagaccggctatcgatatgccgtattgcgtttcgtggaccatggccccggcgtgccgcctgaatcgcgctcgaagattttcgaacgcttctacaccgcggacccatcccgtgcccgcgaaaagggcggtactggtttgggcatggccatcgcgcaatccgtggtcaaagcacatcacggctttatctgcgccaccggcaccgatgggggaggcctgaccttcaccgtggtgctgccgattgagcagatcgccgctcctgagcccaagcagtccaccggcaaaaccaaggacgccaaacagaagacttcttggttcagctctgagcgtaagactcaggcgactcagcccaaagcgtgaggtcaagccctgtgaggtaaactgtaccttcggtttactatttccattgaggattaaggaaggttcgcaatgcccaccggtcgagttcgttggtttgacgcagccaagggttatggcttcatcaccagtgaggaaggcaaggacgtgttcctgccggctcaggccctacccactggcgtcaccacgttgcgcaagggggccaaggtggagtattccgtggtggacggccgtcgtgggccgcaggccatggatgttcgcctgatcgcctccgctcccagcttggtcaaggccacccgcccgaaggcggatgacatggccgccatctgcgaggacctcatcaagatgcttgatgcggccggcaatacgttgcgccgtcaccgttacccatctgctgccgacagcaaaaagctggccacgttgctgcgtgcggtggcggatcaattcgatgtacaggactgatctgatctatgactgataccaccgaaaccatcgtgacaccggaccctcacgccatcgcgcgcgccgtgctgctcgaagtggccgacgagagcgatcaggtgggggatttcgttacctcatatgatcttgaggatcacgtcactgacttccgcttcgccgcgaatatccgtggctacgaaggatggcaatggtcggtgacgctgtatcacgacgaggagatcgactcctggaccgtcaacgaatcctccttgatctccactgaggacgcgttgatgccgcccaagtggattccttggaaggatcgccttgagcccacggatctggctcccaccgattcgatcggtaccgatccggatgacgagcgcatcgaggaaggcgaagtcgaggaatcctcgctgcaggatgtcaacgacgccgtcgagaccttccgactgacccgccgtcacgtgctgacctcgctgggccgcgcgcaggctgccaagcgctggtatgaaggtccgcgaggccccaaagcgttgagcaccaaaaccgccgaaggcaacctgtgctccacctgtggtttcttcgtgccgctcgcgggcgagctcgaccgtatgttcggcgtgtgcgccaacaagtggagtccggatgacggccgcgtggtttccttggaccacggctgcggcgagcattccgaaatcgaacctcccgagccaagccagctgtgggtgcagtccaagcctgctttcgacgatctgcatatcgacgtggtggccaaccgtccgcgcaagcaggagccggctgcccaggacgaggccgaaggcgaaaccgacaagacaggcgagcctgccggtgatgatatcgaagcacagaagactgttgacgggaacgccgtcgatactgagcccgctgaggaggcggcaggcgacgggacctcgcaatcccagtctgccggtgatgagtccgttgcacagaacgtatctgattccgttgtggatgttgatgcagctgattccagcgacgattccaagtctgatgctgacgacgaggccaccgaagaggatatcctcaacaacaccgttgccgatgatgatgaggatgatgagatggacgacgaagaggataacgttcgtccttcagacgatgtgactcctgaactcgaaaccgtcatcgatctcattgagcagctgcgccagaacagggcggacgaggaataaactccgaattccgcgaaaagtagatggtgatggggttgcaggaataaagaattcctccctgcaagcccatcaccgaggcgtgcgttagcactccatgccgattttggtgaatcagtgcaccacggtcaccgtgcattcggcgaagttcacgatctggcgggacaccgatcccaggaagtgcgcgtccagcccggacaggccacgcgagccgaccaccagatggctggcgtaccgcgaggccgcgatcagacctttggatgcgggaatatggaaggcgtttgtgctgaccttgacgccatcgggaatccgggccttggccatcagctcagagagaatctcctcggcgcggcgctgaccgaccttcaccggtgccacggcgttctcatagcccggaataacgcccaaatccttgagctgccagcagaacatcacatgcagcggcgcgtcgtgcagtcgtgcttcttccagtgcgaaatcgaacgcacgg

SEQ ID NO: 3 - DNA, 12478 bp, Bifidobacterium longum
attcctttcttggctcccctctctgagggaagccagggtgcagctcgaacgtctgttcgaacactggggtactataggacgttatgagtgagaacgatttgtttggggcggctgatgcgccggagtcgatgacgcggccgttggcggtgcgcatgcgtccacgcactttggacgaggtgattggccaaacgcaggtgttgggccagggctcgccgctgcgccgcctcgccaatccggccagcaaaggcagtctcaccgcgccgagctccgtaatcctgttcggccctccaggcgtcggcaaaaccacgctcgccaccatcgtggccggccaatccggccgcgtgtttgaggaactctccgccgtgacatccggcgtcaaagacgtgcgcgacgtactcacccgcgctcacgaacggctcgtgtcacgcggccaggaaaccgtactgttcatcgacgaggtacatcgcttctccaaatcccagcaggatgcgctgcttcccgccgtcgaaaaccgcgacgtgacctttatcggcgccaccaccgagaacccgagcttttccatcatcaaaccgttgctctcccgttcggtggtcgtcaagcttgagtcattggagcctgatcagctcatcgaactggtgcagcgcgcgctgactgatgaccgcggtctcaggggcgaggtcaaagccacggacgaagccatcgccgacatcgtgcgcatggccggtggtgacgcacgcaagagtctcacgattctggaggccgcggctggcgcggtcactggcgacgaagcccgcaaaaagggcgcacgccgacccatcatcacccccgaaatcgtggctacggtaatggacaccgccaccgtgcgctacgacaaagacggcgatgaccattacgacgtgatttccgcattcatcaaatccatgcggggctccgatccggatgccgccatccattatctggcacgcatgctgaaagcgggtgaagatccgcgcttcatcgcccgtcgcatcatgatcgccgccagcgaggaagtcggcatggccgccccgcagattttgcaggtcaccgtggccgcggcgcaggccgtggcgctggtcggcatgcccgaggcacgtattattttggccgaagctacgattgccgtggccaccgcgcccaaatcaaacgccagctataacgccattaaccaggcgctcgcggacgtggatgccggcaagatcggcgctgttccgctgtatttgaggaacgcccccaccaagcttatgaaagagtggggcaaccacgagggctacaaatatgcgcacgattggcccggtgccgtggcgccacaggaatatatgcccgaagagctgcgtggcaccgagtactaccatcccaacgatcgcggctatgaacacgaagtcagccagcgtcttgccaagattcgcccgatactgcatggtggggaacccgaacagaaatgaacataggggagtaaagtgtaacgcaacaataacagtggaaaaaggggaggcaaacaccatggcgaccatcttcatcgtggacgatgatcaggccatcggcgaaatgcttagtcttgttttggaaaacgagggattccagactgtgacctgcctggacgggctgagggctgtggaaatgttccccatcgtcaagcccgatttgattctgcttgacgttatgctccccgggctggacggcacggaggtggcccgccgcatcagagctacctctaatgtgccgattatcatgctcaccgccaaatcagacacacttgacgtcgtggccggcttggaagccggtgccgacgactacgtgcccaagcccttcaaagtagccgaactgctggcgcgtatccgtgcacgcttccgcatcgccaagccggccgccgaagacggtgccaccggtggcgcaagcggtggcaatgcaaacgtcaaccatctggaacgcggacccatcgtcatcgaccgtttggaacacaccgccaccaaggatggcaaggacctgaatctcactccgatggaattcgagctgctgttcatgcttgcggccgccgccggcgaggccatcagtcgttccagcctgctcaagaatgtctggggatacgagaattccggcgatacgcgcctggtcaatgtgcatgttcaacgccttagggccaaagtggaggacgaccccgagaacccgcaaatcgtgcagaccgtacgcggtattggctacaagttcgtcactcctgaacaatgaacctgcgccctcgattcagcctcaaacgcttgctgcgccacggacgcgctgaagttcgtcgctcactgcaggcccgcaccgtggccctcaccgtgattcttacgctggccgtggccatcgtcttctctggcgtatcgatggtatcagtgcgcgcctcgctgcttacccaaatcacttcgcagtcgcgtgccgactattcaaacatggtgcagcaggcccaaaccagtctggacgcggccgatgtctccaccgcagtgcagctccaacagctggtcaatgacttggcgtcctcgttgcaatccgaggggtcctccaacctgataggtgtgtatttgtggagtcgtgataccaactcgcgtgccatcatccccgtatccaccgaacccagctatcaaagtctgatttctgacgacatccgttcatccgtggcttccgacttggatgacagcgtgttctatcagccggtcgaaattcccggtgattccggcatgccgggcagcgggacgcccgcagcggtgctcggcaccgtattggactttggcgtggccggcaacctcgaattcttcgccatttattcgtacacgttccagcagcagtccttgacgcagattcagctcagtctggtggtcatctgtgcgttgctgtccatcgtggtgggcgtagtaatctggctggtgattcgtggcatcgtgcgcccgattgaacgagtggcagcggcctcggaaacactggcctccggcaatctcgatatgcgcgttaccgtggatcgtaaggatgagcttggtgtgctgcagcaatccttcaatacgatggctgatgcgctcaaccagaagatcgatgagctggaggaagcaagcgttttccagaaacgatttgtgtccgacgtcagtcatgaattgcgtaccccggtgaccaccatgcgtatggcttccgacctgctcgaaatgaaaaaagacggcttcgacccctccaccaagcgtacggtcgaactgttggccggccagatcagtcgattccaagacatgctcgccgatctgttggaaatctcccgctacgatgccggttatgcggcactcgaccttgtggaaaccgatttatgcgaaccaattgaaacggcagtggaccaagttgacggcatcgcccaagccaaacgggtgccgattcacacgtatctgcccaatgtgcaggttttgactcgtatcgattcgcgtcgagtgattcgcatcgtcagaaatctgttggccaatgccgtcgatttcgctgaggaccggccgatcgaagtgcgcgtcgcggccaaccgcaaagccgtggccatcagcgttcgcgactatggcgttggtatcgacgaagacaaagtcgctcatgtattcgatcgattctggagaggcgacctttcacgttcacgcgttaccggcggcaccggattgggcctgtccatcgccatgactgatgccttgctgcaccacggtagtatccgtgtacgttcggcagtgggggagggcacctggttcttggtattgctgccgcgtgaccccgaccaaggtgaggtggcggacgctgaactgccggtgaattttgcttctgaaacgccggatgacctgcgtgttaccggtgggtttggtgtggccaccagccaagtcacacatgattatcatgaggttcgccgcgacacgatgatggggaggccactatgagacgagtgaccagaacgattgccgccgcaggcgcggccatagcctgttgcatcacgatgacggcatgctcaagtccgttcgatttgccgattagcggctcggtgcagactctggcaccggtggaacagcagacccagcgtgtttacaccaatcctcaggggcctgcggacgatgcacagccggaaaccattgtcaaagggttctatgacgctatgcctgctggcgtgcagttccggatggctattcgcgtggctcgcgaatttcctgactggcttcggctttctgccgggtggaatggagattctgcggcactggtatacagcggcacccctgatttccgccgacgcgccaacaccataagcgcgccacaaggtgcggaaagctcactgattgtggaagtggaacttcaggtggtgggttccttggattcgcatggcgtatacacgccgtcaaacagcacccaaacgcgcaggcttccctatacgctgatgaagaaaagcggccaatggcgcatctcgtcattggaaagcggcgtggtgatttccaccgcagattttgaacaggtgttccgccaagtatccgtgtatcaggtgagtacttccggcaagcaacttatcccggatattcgttggttgagctggcgcaactggcgcacccaggcagtcggtgaagtgctctcggatgcaccctcctggttggaaggagtgctgcgaggcgccggcttgtctaccatcaaactcgcggtcgacagcgtgccggtgaagaacaatgtggtggagatacacctcaacagcggtatcaatgcgttgaacgaggaagaacgaggcctgctggtacatcgcatccgtctgactatgggcgacggcaatgccgaatacgcgctgaggattaccggcgatggagtggactattccgatgccgacgcaaatgtgaaacttactaccgagcagccgacagcgggcgtatacacgttgaccggtggtcatatcgtttccctggccagctccagtccgttgcgtgtaggggaggcccccggatatgacgatgctcggggcttcgttttttcctcatccggcggcgcggtgttgcgtgcggacggcgtggtcgaatgcctgaaatctgatggtgcgtcctgtggggtgatgttctccggcgagccgatgcgatcgattaccgaaggtctggatggcgaagtatgggctgtatccgagaacgggcgcgaattgcatgtgtcagatgggggcaaggaaaccgatctgaagcttgattggcttggtgctgccgacagcattgtggcattggcggtttctccggaagggtgccgattggcgttggccagtcgagggcgaggaccacgaacgggcgttgatgatgaccggtgtggcgccgcaaacgggtgataaaaacactgagcggtctgagcaaaaggccggccacccaagtgagtgtgctcagggcacggttcacccaatgctcaaccgttctacaatgatctgaatctggtgtacgccaccacacctcctgagggaaacagcgaacaacaagaggcatggcgtcaaatggcaccaggcccggccaatgcgcagcgtttacccaatggaatcataacgtcgatggcatcggggcagatcagcctgtcccgtcgcctggccattgtggacgatttgggtattgtgcgctccgtttccggctcactcgacggttcctggaccatcgccgatagccaggtcactgccctcggtgcgcagtagatggtataagaaaaacggtgttcataataacttttcttgtgttgtgatgcggcggctttgtcgctcggctgacgagcggcattgccgccgtgcaaccacctcttattatggcttaataacaaggcaaaacggcgctgtgaagccgttttttgaactactgttatcaaactgttataaacctgttatcaaactgttatcgcatgcgattgaacatttggcctgtgagcgtcacactagagattgtcgatgacgaacagtcaccaaacaggatgaagatcatccaaaaaaataaacactgctcgaggaggagtatttatgaagaattggaagaaggccattgccctcgttgcttctgctgtgcgcttgtcagcgttgccgcatgcggttccagcaacgcaggtggcagctcggactccggcaagaagacggttggcttcgttgctgtgggccctgagggcggcttccgtaccgccaacgagaaggacattcagcaggcattcgaggatgccggctttgacctgacctactctccgacccagaacaacgatcagcagaagcagattcaggcgttcaacaagttcgttaacgacgaagtcgacgccatcatcctgtcctccaccgaggattccggttgggatgactccctgaagaaggccgctgaggctgagattccggtcttcaccgttgaccgtaacgtggacgtcaaggacgccgaggccaagaaggccatcgttgctcacatcggaccgtccaacgtctggtgcggcgagcaggctgccgagttcgtgaacaagaacttcccggatggcgccaacggcttcatcctcgaaggccctgccggcctgtccgtggtgaaggatcgtggcactggttgggacaacaaggttgcctccaacgtcaaggttcttgagtcccagtccgctaactggtccactgatgaggccaagaccgtgaccgctggtctgctcgacaagtacaagtccgacaacccgcagttcatcttcgctcagaacgacgagatgggcctcggtgccgctcaggctgttgacgccgccggcctcaagggcaaggtcaagatcatcaccatcgacggtaccaagaacgctctgcaggctcttgttgatggcgacctctcctacgtgatcgagtacaacccgatcttcggtaaggaaaccgctcaggccgtcaaggactatctggatggcaagaccgttgagaaggacatcgagatcgagtccaagaccttcgacgccgcctccgccaaggaagccctggacaacaacacccgcgcctactgataagtctgctgcgactcattgataactggaccaaataattgatgtgatggtgtgagaaggatgttccttccacgccatcactcatgtgtggctcaaatatcacaaaacgataacatctcattctcgtttaaggcaaagacatgacagataaaaaccccatcgtcgtaatgaaaggcattacgattgaattcccgggcgtcaaggccttggatggtgttgatttgactctctacccgggtgaagttcacgccctgatgggtgagaacggtgcaggcaagtccaccatgattaaggctctgaccggtgtgtacaagatcaacgccggctccattatggtggacggcaagcctcagcagttcaacggcaccctcgacgcacaaaacgccggtatcgccaccgtgtatcaggaagtgaacctgtgcaccaacctttccgtcggtgagaacgtgatgctgggccacgaaaagcgcggccccttcggcatcgactggaagaagacccacgaggccgctaagaagtatttggcacagatgggcctcgaatccattgacccgcacactccgctgagctccatctccatcgctatgcagcagctggtcgccatcgcccgcgctatggttatcaacgccaaggtgctgattctcgatgagccgacctcttcgctggatgccaacgaggtcagggacctgttcgcgatcatgcgcaaggtgcgtgactcgggcgtggccatcctcttcgtctcccacttcctcgatcagatttatgagattaccgatcgtctgaccattctgcgtaacggccagttcatcaaggaggtcatgaccaaggacaccccgcgcgacgaactcatcggcatgatgattggtaagtccgccgccgagctgtcccagattggtgccaagaaggctcgccgtgaaatcacccctggcgagaagccgatcgtcgatgtcaagggcctcggcaagaagggcaccatcaacccggttgatgttgacatctacaagggtgaggtcgttggcttcgctggcctgctgggctccggtcgtaccgagcttggtcgactcctgtatggtgccgacaagccggattcgggtacctacacgctcaacggcaagaaggtcaacatctccgatccgtacacggctttgaagaacaaaatcgcgtactccaccgaaaaccgtcgtgatgagggcatcatcggcgacctgaccgtccgccagaacatccttatcgccctgcaggcaacgcgcggtatgttcaagccgattcccaagaaggaagcggacgccatcgttgacaagtacatgaaggaactcaacgttcgtcccgccgacccggatcgcccggtcaagaatctctccggcggcaaccagcagaaggtgctcattggccgttggctggccacgcaccccgagctgctgattctggacgagccgacccgtggtatcgatatcggtgccaaggctgaaattcagcaggtcgtgcttgacctggcttctcagggcatgggcgttgtcttcatctcctccgagcttgaagaggtcgtgcgtctgtccgacgacatcgaggttctcaaggaccgccacaagatcgcagaaatcgaaaacgacgacaccgtctctcaggcgaccatcgtcgaaaccatcgctaacaccaacgtaaacaccggaaaggaggcatgagatggctgaaaaggcaaaagccgagggcaacaactttgtcaagaagctgctgagcagcaacctgacctggtcgatcgtcgcattcattcttctggtcatcatctgcaccatcttccagcatgacttcctggctttgagctggaacagcaacaccggtggtctggccggcccgctgatcaccatgctccaggaatctgcccgatacctgatgattgcaaccggtatgaccttggttatctccaccgccggtatcgacctttcggtcggttccgttatggcagtggcaggtgccgccgccatgcagaccctgtccaatggcatgaacgtgtggctctccatcctcatcgccttggctgttggtctggccattggctgcgtcaacggcgctctggtttccttcctgggcctacagccgttcatcaccaccctgattatgatgctcgccggccgtggtatggccaaggtcatcacctccggtgagaacaccgacgcctccgcagttgctggcaacgaaccgctgaagtggttcgccaacggcttcattctgggcattcccgccaacttcgtcatcgccgttatcattgtgattctcgttggcctgctgtgccgcaagaccgctatgggcatgatgattgaggccgtgggcatcaaccaggaagcctcccgtatgaccggtatcaagccgaagaagatcctcttcctcgtctacgcgatttccggcttcctcgcggccatcgctggtctgttcgccaccgcatccgtgatgcgtgtcgacgtggttaagaccggtcaggacctcgaaatgtacgccattctggcagtcgtcatcggcggtacttcactgctgggtggtaagttctccctcgccggctctgctgtcggtgctgtaattatcgccatgatccgcaagaccatcatcaccctgggcgtcaacgccgaggcaactccggccttcttcgccgtcgttgtgattgtgatctgcgtgatgcaggctccgaagattcacaacctgagcgcgaatatgaaacgcaagcgcgcgctcaaggctcaagctaaggcggtggcagcaatgacaacagctacggcaaacaaagtgaaggctcccaagaagggcttcaagctcgatcgtcagatgatcccgaccctcgcggccgtggtgatcttcatcctgatgatcatcatgggtcaggcgttgttcggcacctacattcgactgggcttcatctcctccctgttcattgaccacgcctacctgattattctggctgtggccatgaccctgccgattctgaccggtggtatcgatttgtctgtcggtgctatcgtggccatcaccgcagtcgtcggcctgaagctggcgaacgccggcgtgcccgccttcctggtcatgatcatcatgctgctcatcggcgctgtgttcggcctgctggccggcaccttgatcgaggaattcaacatgcagccgttcatcgcgaccctgtcgacgatgttcctggcccgtggtcttgcctccatcatctccaccgactcgctgaccttcccgcagggcaatgacttctcgttcatctccaacgtgatcaagatcatcgacaatccgaagatctccaacgatctgtccttcaacgtcggcgtgatcatcgcactggtggttgtggtcttcggctacgtcttcctgcaccatacccgcaccggacgcaccatctacgccatcggcggctcccgttcctccgcggaactcatgggtctgccggtcaagcgcacgcagtacatcatctacttgacctctgcgactctcgccgccctggcctcgatcgtgtacaccgcaaacatcggctctgccaagaacactgtgggtgttggctgggagctcgacgccgttgcctccgtggtcatcggcggtacgatcatcaccggtggcttcggctacgtgctcggctccgtgctcggctctctggtccgctccatcctcgatccgctcacctctgacttcggtgtgccggccgaatggaccaccatcgttatcggtctcatgatcctcgtcttcgttgtgcttcagcgcgcggtgatggcggtcggcggagataaaaaatagcgggcttgcccgctcggtctccgcaccgaatacgacacacaccataactgaataggcgggatcagcccggctgctgtgatggcaaggtcacagcagccggcactgcccgccaaagctttctttctccttttcttcctttcattagcgtgtgggcgtgccccggggttctcccggggcacgccccacacgtgtttggaaagctgggactctattgatgatcggatttatgcacgcttacgtgccgcgcgaatgcggaacgcggtggccagacagcaccagccaatcaggcagataaccgtcatcgtggcaaacgtccatgtgccgccgacggcagtggcgtgagcaaactcagcggtgccttcttctcctgcgccggcctgagccacggaaagaatcgtggagaacagtgcggtaccggcagcgccgccaaactgaagacacgtgttgaaaatggcgttgccgtccggcttgaactcgggagagacctccgacaggccactggtcatcacattcgcattgccgatggaatagaacagaccgaaaatgaaatagaatccggcgagcagcaagggcgtaagctgcatcgagaacaccagcataagcactgggcccaaaatagcgatgccgatcggaatcagaatcggcttgaccgcgccgaacttatcgtagaaccagccgccaagcggggcacagacagcgccgaccagtgctccgggcagtaccagagagccggccacaaatgccgtggtaccgagcgagagctgtgccacattggtgatgacatagccgtagccgatggcgaccaatggcagcagcagatatgcgcaggcgtgtaacagtacggcgggatccttgagaatgccaaggcgcagcagcggggagaaggcacgcttggaactcatggcgaacacgatcaacaaggcgaggcccgcaatcagactgacaatggcgataatgccggagcgtgtggctgacttgccggacaccgccgcactgatggccacgccaccttggttgagcgccagcaccagaccgaccagcgcgacaacaatggacccaagctgaatcgggtcaagataggcagcctcggtcggtgtcttctgctcaacaggccgttgatcgaaccgcgcaccagacggttaccgaagttgtggaacgggcgcttgttctcttggaagtaggtggagctcaagcggtcgccggttaccatgtcgtatccttcggcgatcttctcgaccatggcgggtgccgcgtcggctgggtaggtgtcatcgccgtcggccatgacgtacacatcggcgtcgatgtcttcgaacatggctcggatcacgttgcctttgccttggcgtggctccttgcggacgatggccccttccgccgccgcgatctcggcggtgcggtcggaggagttgttgtcgtacacgtagatgtccgcctgcggcagcgcgattttaaaatcacgcacgaccttgccgatggtgacttcttcgttgtagcaaggaagaagtaccgcaacggaaacgttcgaattctcaggcatgcactccactatatctcttgtactggtctgcatggggaatggattataggtgatgctgcatacttgaagcaaatggatttatcgattcacattaattatcacgagtgtagcgtgacgaagtgttcgtcttgaatgccgaatcgcttttgaaataatcgttatagctgatctccatgt

SEQ ID NO: 4 - protein, 348 aa, Bifidobacterium longum (bikC)
MVSGRGAQSFLTLADPDRHRCGDARSLSRRRSARGTDAPAHREWAGTGRRRPHQHCRASGPICRPPRLFSRQPRCPGDRGRWRRGHCHCRPRSGAQTRGTIPRRRNRKPRRDGGYRRPGRSGDASRTAGRKPIDRIQREAVNVAPYWLPCWEQRAAGVEHMVGRMFSAQEEERRRVSQELHDGVAQTATALARILEGVGEGQTEALPAAERDRLAGIARALVRELRAVIGGLRPTLLDDLGLQAALRSLADGLEEDGYQVSFCMADDASRLSPTVEIALFRVAQEAIANVRKHAGGPCAVAIALRIETGHLRLQIQRQRPRAERSAWTLAQEPGRPVAMSASTSCMNE

SEQ ID NO: 5 - protein, 214 aa, Bifidobacterium longum (birC)
VAGPRILIVDDHQLAREGLRAVLAQSGVNVVGVASSGEEAIDQVRLLHPDVVLMDVRLGSGIDGLEATRRIAALDTATRILMLTLHDMPAYVREALAAGAAGYVLKDTAIGDLIAAIDQVMAGNSAVPLALVNAAMRAPALPQRDADISRVLTSREQEVVALVARGLTNKEIARELAISPATVKAHVERVIGKLGVADRTQAAVLAAQMRPAGL

SEQ ID NO: 6 - protein, 448 aa, Bifidobacterium longum (bikB)
MPTMPPSSRARAPWRILPVTFQQNGKDRAVVYIGLSLADQIDTVNTLTRYCIVVGIAVVLLGGSLSTLIIQHTMTPLKRIEKTAAKIAAGDLSQRIPSAPENTEVGSLAASLNSMLTRIESSFHEQEETTDKMKRFVSDASHELRTPLAAIHGYAELYKMQRDMPGALERADESIEHIERSSQRMTVLVEDLLSLARLDEGRGIDMTGTVKLSSLVTDAVDDLHALDPDRAVRRMQISLEPARDLNHPAEFSLAEGDWPEVVLPGDASRLRQVVTNIVGNIHRYTPADSPAEAALGVMPAAIDPRQLARMPASDASMRRFIDAAEVGASMQTGYRYAVLRFVDHGPGVPPESRSKIFERFYTADPSRAREKGGTGLGMAIAQSVVKAHHGFICATGTDGGGLTFTVVLPIEQIAAPEPKQSTGKTKDAKQKTSWFSSERKTQATQPKA

SEQ ID NO: 7 - protein, 225 aa, Bifidobacterium longum (birB)
MSKPIEASIVVVDDEPSIRELLVASLHFAGFEVNTAASGSEAIEVIEKVQPDLIVLDVMLPDIDGFTVTRRIRQEGINAPVLFLTARDDTQDKIMGLTVGGDDYVTKPFSLEEVVASSAPFCAVPANRVEDDPIRQSAGTLEINEDSHDVTRAGQPVDLSPTEYKLLRYLMDNEGRVLSKAQILDHVWQYDWGGDAAIRRISTSPTCARKSTASRSTTAKAASAR

SEQ ID NO: 8 - protein, 565 aa, Bifidobacterium longum (bikA)
MNLRPRFSLKRLLRHGRAEVRRSLQARTVALTVILTLAVAIVFSGVSMVSVRASLLTQITSQSRADYSNMVQQAQTSLDAADVSTAVQLQQLVNDLASSLQSEGSSNLIGVYLWSRDTNSRAIIPVSTEPSYQSLISDDIRSSVASDLDDSVFYQPVEIPGDSGMPGSGTPAAVLGTVLDFGVAGNLEFFAIYSYTFQQQSLTQIQLSLVVICALLSIVVGVVIWLVIRGIVRPIERVAAASETLASGNLDMRVTVDRKDELGVLQQSFNTMADALNQKIDELEEASVFQKRFVSDVSHELRTPVTTMRMASDLLEMKKDGFDPSTKRTVELLAGQISRFQDMLADLLEISRYDAGYAALDLVETDLCEPIETAVDQVDGIAQAKRVPIHTYLPNVQVLTRIDSRRVIRIVRNLLANAVDFAEDRPIEVRVAANRKAVAISVRDYGVGIDEDKVAHVFDRFWRGDLSRSRVTGGTGLGLSIAMTDALLHHGSIRVRSAVGEGTWFLVLLPRDPDQGEVADAELPVNFASETPDDLRVTGGFGVATSQVTHDYHEVRRDTMMGRPL

SEQ ID NO: 9 - protein, 240 aa, Bifidobacterium longum (birA)
MATIFIVDDDQAIGEMLSLVLENEGFQTVTCLDGLRAVEMFPIVKPDLILLDVMLPGLDGTEVARRIRATSNVPIIMLTAKSDTLDVVAGLEAGADDYVPKPFKVAELLARIRARFRIAKPAAEDGATGGASGGNANVNHLERGPIVIDRLEHTATKDGKDLNLTPMEFELLFMLAAAAGEAISRSSLLKNVWGYENSGDTRLVNVHVQRLRAKVEDDPENPQIVQTVRGIGYKFVTPEQ

SEQ ID NO: 10 - DNA, 975 bp, Bifidobacterium longum (bia A)
ttggaagaaggccattgccctcgttgcttctgctgtgcgcttgtcagcgttgccgcatgcggttccagcaacgcaggtggcagctcggactccggcaagaagacggttggcttcgttgctgtgggccctgagggcggcttccgtaccgccaacgagaaggacattcagcaggcattcgaggatgccggctttgacctgacctactctccgacccagaacaacgatcagcagaagcagattcaggcgttcaacaagttcgttaacgacgaagtcgacgccatcatcctgtcctccaccgaggattccggttgggatgactccctgaagaaggccgctgaggctgagattccggtcttcaccgttgaccgtaacgtggacgtcaaggacgccgaggccaagaaggccatcgttgctcacatcggaccgtccaacgtctggtgcggcgagcaggctgccgagttcgtgaacaagaacttcccggatggcgccaacggcttcatcctcgaaggccctgccggcctgtccgtggtgaaggatcgtggcactggttgggacaacaaggttgcctccaacgtcaaggttcttgagtcccagtccgctaactggtccactgatgaggccaagaccgtgaccgctggtctgctcgacaagtacaagtccgacaacccgcagttcatcttcgctcagaacgacgagatgggcctcggtgccgctcaggctgttgacgccgccggcctcaagggcaaggtcaagatcatcaccatcgacggtaccaagaacgctctgcaggctcttgttgatggcgacctctcctacgtgatcgagtacaacccgatcttcggtaaggaaaccgctcaggccgtcaaggactatctggatggcaagaccgttgagaaggacatcgagatcgagtccaagaccttcgacgccgcctccgccaaggaagccctggacaacaacacccgcgcctactga

SEQ ID NO: 11 - DNA, 2091 bp, Bifidobacterium longum (bia C)
atggctgaaaaggcaaaagccgagggcaacaactttgtcaagaagctgctgagcagcaacctgacctggtcgatcgtcgcattcattcttctggtcatcatctgcaccatcttccagcatgacttcctggctttgagctggaacagcaacaccggtggtctggccggcccgctgatcaccatgctccaggaatctgcccgatacctgatgattgcaaccggtatgaccttggttatctccaccgccggtatcgacctttcggtcggttccgttatggcagtggcaggtgccgccgccatgcagaccctgtccaatggcatgaacgtgtggctctccatcctcatcgccttggctgttggtctggccattggctgcgtcaacggcgctctggtttccttcctgggcctacagccgttcatcaccaccctgattatgatgctcgccggccgtggtatggccaaggtcatcacctccggtgagaacaccgacgcctccgcagttgctggcaacgaaccgctgaagtggttcgccaacggcttcattctgggcattcccgccaacttcgtcatcgccgttatcattgtgattctcgttggcctgctgtgccgcaagaccgctatgggcatgatgattgaggccgtgggcatcaaccaggaagcctcccgtatgaccggtatcaagccgaagaagatcctcttcctcgtctacgcgatttccggcttcctcgcggccatcgctggtctgttcgccaccgcatccgtgatgcgtgtcgacgtggttaagaccggtcaggacctcgaaatgtacgccattctggcagtcgtcatcggcggtacttcactgctgggtggtaagttctccctcgccggctctgctgtcggtgctgtaattatcgccatgatccgcaagaccatcatcaccctgggcgtcaacgccgaggcaactccggccttcttcgccgtcgttgtgattgtgatctgcgtgatgcaggctccgaagattcacaacctgagcgcgaatatgaaacgcaagcgcgcgctcaaggctcaagctaaggcggtggcagcaatgacaacagctacggcaaacaaagtgaaggctcccaagaagggcttcaagctcgatcgtcagatgatcccgaccctcgcggccgtggtgatcttcatcctgatgatcatcatgggtcaggcgttgttcggcacctacattcgactgggcttcatctcctccctgttcattgaccacgcctacctgattattctggctgtggccatgaccctgccgattctgaccggtggtatcgatttgtctgtcggtgctatcgtggccatcaccgcagtcgtcggcctgaagctggcgaacgccggcgtgcccgccttcctggtcatgatcatcatgctgctcatcggcgctgtgttcggcctgctggccggcaccttgatcgaggaattcaacatgcagccgttcatcgcgaccctgtcgacgatgttcctggcccgtggtcttgcctccatcatctccaccgactcgctgaccttcccgcagggcaatgacttctcgttcatctccaacgtgatcaagatcatcgacaatccgaagatctccaacgatctgtccttcaacgtcggcgtgatcatcgcactggtggttgtggtcttcggctacgtcttcctgcaccatacccgcaccggacgcaccatctacgccatcggcggctcccgttcctccgcggaactcatgggtctgccggtcaagcgcacgcagtacatcatctacttgacctctgcgactctcgccgccctggcctcgatcgtgtacaccgcaaacatcggctctgccaagaacactgtgggtgttggctgggagctcgacgccgttgcctccgtggtcatcggcggtacgatcatcaccggtggcttcggctacgtgctcggctccgtgctcggctctctggtccgctccatcctcgatccgctcacctctgacttcggtgtgccggccgaatggaccaccatcgttatcggtctcatgatcctcgtcttcgttgtgcttcagcgcgcggtgatggcggtcggcggagataaaaaatag

(19) United States
(12) Patent Application Publication
(10) Pub. No.: US 2005/0037395 A1
(43) Pub. Date: Feb. 17, 2005

(54) Product

(76) Inventor: Douwe Van Sinderen, Cork (IE)

Correspondence Address: JACOBSON HOLMAN PLLC, 400 SEVENTH STREET N.W., SUITE 600, WASHINGTON, DC, 20004, US

(21) Appl. No.: 10/876,542

(22) Filed: Jun. 28, 2004

Related U.S. Application Data
(60) Provisional application No. 60/482,873, filed on Jun. 27, 2003.

(51) Int. Cl. C12Q 1/68; C07H 21/04; C12N 9/12; C12N 15/74

(52) U.S. Cl. 435/6; 435/69.1; 435/194; 435/252.3; 435/320.1; 536/23.2

(57) ABSTRACT

An isolated Bifidobacteria DNA fragment comprises nucleic acid selected from sequence ID No. 1, sequence ID No. 2 or sequence ID No. 3. A protein having sequence ID No. 4, sequence ID No. 5, sequence ID No. 6, sequence ID No. 7, sequence ID No. 8 or sequence ID No. 9 is also disclosed as are DNA fragments comprising sequence ID No. 10 or 11 and proteins encoded thereby. A two-component signal transduction system comprises a gene encoding sequence ID No. 4 and a gene encoding sequence ID No. 5, a gene encoding sequence ID No. 6 and a gene encoding sequence ID No. 7 or a gene encoding sequence ID No. 8 and a gene encoding sequence ID No. 9. The Bifidobacteria may be Bifidobacterium infantis UCC35624.

The invention relates to Bifidobacteria and isolated two-component regulatory systems (2CSs).

Bifidobacteria are among the most common genera in the human colon, and have consistently had health-promoting properties attributed to them (13, 14, 17, 18, 23, 54, 55).

Two-component regulatory systems (2CSs) are employed extensively in nature by microorganisms to modify their cellular physiology in response to alterations in environmental conditions (37, 38, 39, 53). A 2CS typically consists of a membrane-associated sensor protein or histidine protein kinase (HPK), which monitors one or more environmental parameters, and a cytoplasmic effector protein or response regulator (RR), which induces a specific cellular adaptive response. The HPK and RR are each comprised of two modular elements. A typical HPK contains an N-terminally located input or sensing domain, and a C-terminal transmitter domain, which is autophosphorylated at a conserved histidine residue in response to fluctuations in chemical and/or physical conditions (sensed by the input domain). This phosphate group is transferred to an aspartate residue on the N-terminally positioned receiver domain of the cognate RR, which in turn alters the activity of the output domain (situated in the C-terminal region of the RR) to elicit an adaptive response (either functioning at the level of transcriptional regulation or by interacting directly with proteins). The transmitter module of the HPK contains a number of conserved residues in addition to the histidine at the site of autophosphorylation. These include an asparagine box, a glycine residue, a phenylalanine box and a glycine-lysine motif, all located toward the C-terminus of the kinase protein. The conserved receiver domain found in RRs contains a strictly conserved aspartate box and a lysine residue which are part of an acidic pocket involved in the phosphorylation event (35, 57).

2CSs have been found in over fifty prokaryotic species to date, and several lower eukaryotic organisms and plants (10, 25, 36, 40). However, there is diversity in both the number and the organisation of these systems. The number of 2CSs in a given bacterial species can vary from four HPKs and five RRs encoded by the entire genome of Haemophilus influenzae Rd (16), to approximately 50 different 2CSs in enteric bacterial genomes (5, 28).

HPKs have been sorted into classes on the basis of the sequence relationships of the residues surrounding the phosphorylated histidine (20). This classification has resulted in the organisation of HPKs into five homology groups (groups I, II, IIIA, IIIB and IV (15)). RRs have been classified into three major groups (classes 1, 2 and 3), based on the phylogenetic relatedness of their receiver module and DNA-binding domains, and four minor groups (classes 4-7) that exhibit output domains with rather unique amino acid sequences (35).

### STATEMENTS OF INVENTION

According to the invention there is provided an isolated Bifidobacteria DNA fragment comprising nucleic acid sequence ID No. 1, sequence ID No. 2 or sequence ID No. 3 or a mutant or fragment or variant thereof.

The invention also provides a DNA fragment comprising nucleic acid sequence ID No. 10 or 11 or a mutant or fragment or variant thereof and proteins encoded thereby.

The invention also provides a protein having sequence ID No. 4, sequence ID No. 5, sequence ID No. 6, sequence ID No. 7, sequence ID No. 8 or sequence ID No. 9 or a mutant or fragment or variant thereof.

Preferably the DNA fragment or protein is isolated from the probiotic genus Bifidobacterium. Most preferably DNA fragment or gene is isolated from Bifidobacterium infantis UCC35624.

The invention also provides a two-component signal transduction system comprising a gene encoding sequence ID No. 4 and a gene encoding sequence ID No. 5 or a mutant or fragment or variant thereof.

The invention further provides a two-component signal transduction system comprising a gene encoding sequence ID No. 6 and a gene encoding sequence ID No. 7 or a mutant or fragment or variant thereof.

The invention further provides a two-component signal transduction system comprising a gene encoding sequence ID No. 8 and a gene encoding sequence ID No. 9 or a mutant or fragment or variant thereof.

In one embodiment of the invention the two-component signal transduction systems are isolated from the probiotic genus Bifidobacterium, preferably from Bifidobacterium infantis UCC35624.

One aspect of the invention provides a protein encoded by a DNA fragment comprising sequence ID No. 1, sequence ID No. 2 or sequence ID No. 3 or a derivative, fragment or mutant thereof.

The invention further provides a method of screening for the presence of Bifidobacteria using a DNA fragment comprising sequence ID No. 1, sequence ID No. 2 or sequence ID No. 3 or sequence ID No. 10 or sequence ID No. 11 or a derivative, fragment or mutant thereof.

Another aspect of the invention provides a method of screening for the presence of Bifidobacteria. using sequence ID No. 4, sequence ID No. 5, sequence ID No. 6, sequence ID No. 7, sequence ID No. 8 or sequence ID No. 9 or sequence ID No. 10 or sequence ID No. 11 or a derivative, fragment or mutant thereof. The Bifidobacteria may be Bifidobacterium infantis UCC 35624.

The invention also provides a method of screening for the presence of Bifidobacteria using a two-component signal transduction system comprising a gene encoding sequence ID No. 4 and a gene sequence encoding sequence ID No. 5, a two-component signal transduction system comprising a gene encoding sequence ID No. 6 and a gene sequence encoding sequence ID No. 7 or a two-component signal transduction system comprising a gene encoding sequence ID No. 8 and a gene sequence encoding sequence ID No. 9. Preferably the Bifidobacteria is Bifidobacterium infantis UCC35624.

Another aspect of the invention provides use of a protein encoded by a DNA fragment comprising sequence ID No. 1, sequence ID No. 2 or sequence ID No. 3 or a derivative, fragment or mutant thereof in the prophylaxis and/or treatment of undersirable inflammatory activity.

The invention also provides use of a protein encoded by a gene comprising sequence ID No. 4, sequence ID No. 5, sequence ID No. 6, sequence ID No. 7, sequence ID No. 8 or sequence ID No. 9 or sequence ID No. 10 or sequence ID No. 11 or a derivative, fragment or mutant thereof in the prophylaxis and/or treatment of undersirable inflammatory activity.

One embodiment of the invention provides use of a protein of the invention or an active derivative, fragment or mutant thereof in the prevention and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

Another embodiment provides use of a protein of the invention or an active derivative, fragment or mutant thereof in the prophylaxis and/or treatment of undesirable gastrointestinal inflammatory activity such as; inflammatory bowel disease such as Crohns disease or ulcerative colitis; irritable bowel syndrome; pouchitis; or post infection colitis.

Another embodiment of the invention provides for use of a protein of the invention or an active derivative, fragment or mutant thereof in the prophylaxis and/or treatment of gastrointestinal cancer(s), systemic disease such as rheumatoid arthritis, autoimmune disorders due to undesirable inflammatory activity, cancer due to undesirable inflammatory activity, cancer, diarrhoeal disease due to undesirable inflammatory activity, such as Clostridium difficile associated diarrhoea, Rotavirus associated diarrhoea or post infective diarrhoea or diarrhoeal disease due to an infectious agent, such as E. coli.

One embodiment of the invention provides use of a protein of the invention or an active derivative, fragment or mutant thereof in the preparation of anti-inflammatory biotherapeutic agents for the prophylaxis and/or treatment of undesirable inflammatory activity.

The identification of these two component systems from Bifidobacterium provides a method of screening for the presence of Bifidobacterium in particular Bifidobacterium infantis UCC35624 in samples using PCR or any other suitable method. The DNA fragments and gene sequences may also be used as tags for tracking Bifidobacteria especially Bifidobacterium infantis UCC35624.

The 2CSs identified from UCC35624 may encode proteins which are involved in host immune signals and may be very important in determining the mechanism of action of Bifidobacteria in particular Bifidobacterium infantis UCC35624.

A deposit of Bifidobacterium longum infantis strain UCC 35624 was made at the National Collections of Industrial and Marine Bacteria Limited (NCIMB) on Jan. 13, 1999 and accorded the accession number NCIMB 41003.

### BRIEF DESCRIPTION OF THE DRAWINGS

The invention will be more clearly understood from the following description of some embodiments thereof, given by way of example only with reference to the accompanying drawings in which:—

FIG. 1 is a schematic representation of the three 2CSs identified on the chromosome of B. infantis UCC35624, and their surrounding ORFs. Arrows represent each ORF with the gene name positioned above. The length of the transcripts identified by Northern analysis are indicated underneath each system by a thin arrow. Positions of promoter sequences deduced from primer extension and/or Northern blot analysis are indicated by. [symbol] The positions of putative transcriptional terminator structures are indicated by

[symbol]

FIG. 2 show the alignment of the genetic organisation of System A from B. infantis UCC35624 with corresponding loci in M. tuberculosis CDC1551 (Accession no. AE007145), M. avium subsp. paratuberculosis (AF10884), B. longum NCC2705 (AE014617), B. longum DJO10A (NZ_AABF01000022) and B. breve NCIMB8807. The names of the genes are indicated within arrows for UCC35624. The percentage identities for each protein-encoding gene as compared to the corresponding ORF from UCC35624 are indicated within the arrows for each genome. The degree of amino acid identity (>90, >80, >70, <70) is indicated by the colour of the arrows (red, yellow, green and blue, respectively).

FIG. 3 is a Northern analysis of Systems A and B using RNA isolated from B. infantis UCC35624 at different O.D. 600 nm values (indicated above each lane). The estimated size of the transcripts are indicated on the right. (a) Transcription of System A using an internal 500 bp fragment of bikA as a probe. Similar results were obtained using probes birA and lipA. (b) Transcription of System A using probe gtpA. Similar results were obtained using probes biaA, biaB and biaC. (c) Transcription of System B using probe bikB. Similar results were obtained using probe birB. Northern blots also revealed a 3 kb transcript for System C (not shown) using probes bikC, birC and bicC.

FIG. 4 is a primer extension analysis of the transcriptional start site of the 11 kb transcript of System A. The assumed ribosome-binding site (RBS) and start codon (ATG) of gtpA are indicated in bold. The transcriptional start site is indicated by a solid triangle, and the name of the gene is indicated in italics over the initial methionine residue. The translated amino acid residues of GtpA are shown underneath the corresponding DNA sequence. The arrow indicates the position of the extension product. Proposed −10 and −35 motifs are boxed.

FIG. 5 is a primer extension analysis of the transcriptional start site of the 4 kb transcript of System A. The assumed ribosome-binding site (RBS) and start codon (ATG) of birA are indicated in bold. The transcriptional start site is indicated by a solid triangle, and the name of the gene is indicated in italics over the initial methionine residue. The translated amino acid residues of BirA are shown underneath the corresponding DNA sequence. The arrow indicates the position of the extension product. Proposed −10 and −35 motifs are boxed.

### DETAILED DESCRIPTION

Very little is known about the molecular biology of bifidobacteria, despite the fact that they are among the most common genera in the human colon, and have consistently had health-promoting properties attributed to them (13, 14, 17, 18, 23, 54, 55). Genetic characterisation of bifidobacteria is essential to define their possible beneficial activities as part of the intestinal microflora, and to explore and potentially exploit any such beneficial properties.

The mechanism of action of the probiotic bacteria UCC35624 remains to be fully elucidated. A number of putative modes have been proposed (13, 14, 51). However genetic investigation of Bifidobacterium species has been very limited, due to a paucity of genetic tools and a relatively low electrotransformation efficiency (generally reported as approximately 10⁴-10⁵ cells per μg of DNA (26, 45)). This transformation frequency does not appear to allow single cross-over recombination for the purpose of gene knockouts (47). Thus it is not possible at this time to attribute in vivo phenotypic characteristics to (the mutation of) any of these systems, and functionality can be proposed only as a result of homology studies.

The invention provides the amino acid sequence of Bifidobacterium longum infantis UCC35624. The invention also provides three two-component regulatory systems (2CSs) isolated and identified from the genus Bifidobacterium.

Information on the genetic organisation and regulation, particularly on systems which act at the interface between host and bacterium, such as two-component systems, provide an invaluable tool for understanding the probiotic properties attributed to Bifidobacterium. Therefore the identification of the three two-component regulatory systems (2CSs), signal transduction systems in this genus, has large therapeutic potential. 2CSs may be of critical importance in the interaction between microbe and host (environment).

Two different methods were employed to maximise identification of 2CSs on the chromosome of B. longum infantis UCC 35624. A complementation strategy (32, 56) resulted in the identification of a single HPK, bikA, using the E. coli mutant ANCC22.

A second, PCR-based strategy was employed which allowed the identification of two 2CSs. A specific set of degenerate primers was designed and optimised for use in Gram-positive bacteria with high G+C % content. Subsequent sequence analysis using the three HPK- and RR-encoding fragments allowed the identification of the three complete 2CSs.

The complementation strategy has various technical limitations, such as the particular mutant strain used, the intrinsic properties of the kinase itself, and the portion of the kinase cloned. All of these factors determine if “cross-talk” or heterologous transphosphorylation is possible. These limitations are possibly exacerbated by the difference in G+C % content between E. coli (typically 48-52% (9)) and bifidobacterial DNA (58%). BikA belongs to the Group IIIA kinases (15) and would therefore be predicted to suppress the phenotypic effect of the HPK mutations in ANCC22 (also Group IIIA HPKs). BikB is also a member of this Class IIIA of HPKs and thus would be expected to have been detected by the complementation procedure. However, when the C-terminal conserved moiety of this kinase was cloned into ANCC22, no phenotypic complementation was observed. Therefore it may be that the specificity of BikB prevents the transmitter domain from participating in heterologous transphosphorylation in this case.

It is expected that the genome of B. infantis UCC 35624 would harbour more than three 2CSs considering the frequency in which such systems occur in other bacterial species.

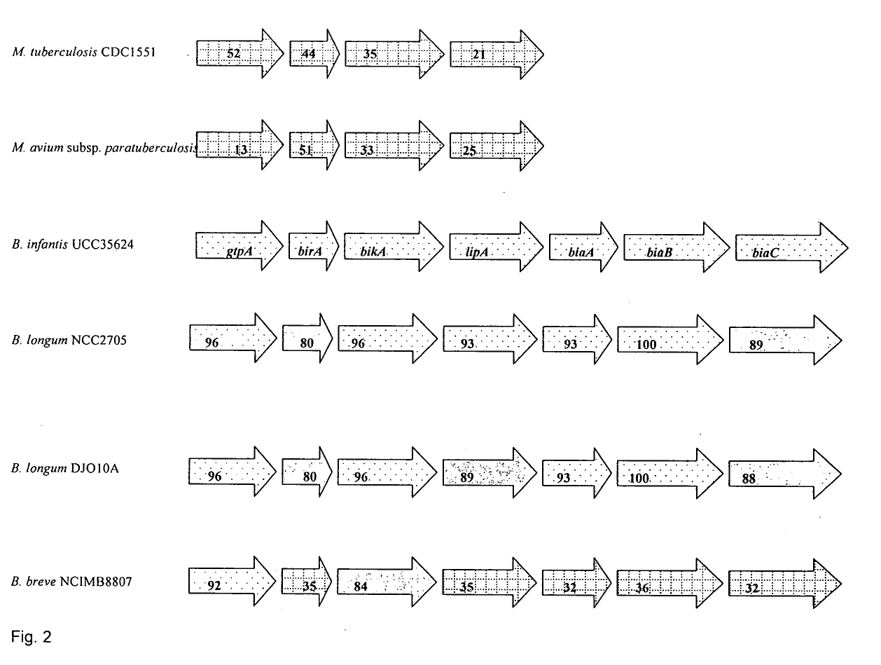

All three operons appear to be typical two-component His-Asp phosphorelay systems. The HPK-RR pair of System A displays significant similarity to a number of putative 2CSs from the related, high G+C % genera Corynebacterium and Mycobacterium. The highest similarities observed (Table 4; FIG. 2) were from the closely related B. longum species DJO10A and NCC2705, and B. breve NCIMB 8807. Downstream of and transcriptionally linked to System A, an ABC transport system was identified, with highest homology to sugar uptake systems. The genetic organisation of the large 11 kb transcription unit of System A is highly conserved across the investigated bifidobacterial genomes. The lipA gene, transcriptionally linked to System A, is consistently located immediately downstream of a 2CS in the bifidobacterial genomes investigated (FIG. 2), as well as in M. avium subsp. paratuberculosis, M. tuberculosis H37Rv and M. leprae TN (Accession no.s AF410884, Z95121 and NC_{—}002677, respectively).

The BirB-BikB and BirA-BikA 2CSs both belong to the OmpR superfamily of 2CSs (15). Homologues of System B can be observed in B. longum DJO10A, B. longum NCC2705, B. breve NCIMB 8807, and M. tuberculosis CDC1551, indicating that this 2CS is widely conserved among high G+C %-content bacterial species. In B. longum NCC2705 in particular, the ORFs surrounding the 2CS display significant similarity (data not shown).

The genetic organisation of System C is different to the other two 2CSs. The gene encoding the HPK in this case is located upstream of its cognate RR-encoding gene. Notably absent in BikC are the transmembrane domains typical of the N-terminus of HPKs. BikC therefore appears to be a cytoplasmic HPK, and possibly responds to an intracellular signal. BikC represents a member of the Group II HPKs, specifically categorised in the DegS subgroup. BirC lacks the C-terminal DNA-binding motif of the OmpR family, and is a member of the NarL/DegU family of RRs (Class 3) (7, 15, 35). System C is of particular interest as it does not appear to have a close homologue in B. longum NCC2705, B. longum DJO10A, or B. breve NCIMB8807 (Table 4), indicating that this 2CS may fulfil a regulatory function not present in (some) other Bifidobacterium spp.

The comparative analysis of the three 2CSs from B. infantis UCC 35624 indicates that two of these have functional homologs in three partially or completely sequenced Bifidobacterium genomes. This is obvious from their high percentage of identity (Table 4), and is further compounded by their conserved gene organisation (FIG. 2). For similar reasons, functional homologs of System A also seem to exist in a variety of Mycobacterium species. The regulatory function of the identified systems is as yet obscure, since no functional studies have been performed for any of the 2CSs. All three systems incorporate a RR protein that contains an effector domain with a DNA binding motif, thus indicating that these systems act to respond to their stimulus by adjusting gene expression. The conserved gene organisation of System A and its co-transcribed genes indicate that such genes may either be targets of the 2CS (several 2CSs are located next to co-transcribed genes, in many cases encoding ABC transport systems (33), they control or may be part of the signal transduction pathway itself. The signals to which these 2CSs respond remain elusive (as they are for most 2CSs known). The HPKs encoded by System A and B are most likely associated with the cytoplasmic membrane and are therefore expected to respond to extracellular stimuli. In contrast, the protein specified by bikC does not appear to contain a membrane-spanning input domain and may therefore respond to an intracellular signal.

From Northern blotting used in the transcriptional analysis of genes from Bifidobacterium spp., each of the 2CSs appear to be growth-phase regulated, a feature which is common in such systems throughout the bacterial kingdom. It is an observed phenomenon in many bacterial species that promoter elements have higher A+T % contents than intragenic DNA. The only experimentally mapped bifidobacterial promoter regions, i.e. the β-gal1 and the lactose permease genes of B. infantis, have a relatively high A+T % content (66% and 73%, respectively (19)). In the present invention the sequences immediately upstream of the TSS of gtpA and birB were found to have an A+T % content of 48%, and 50% in the case of birA.

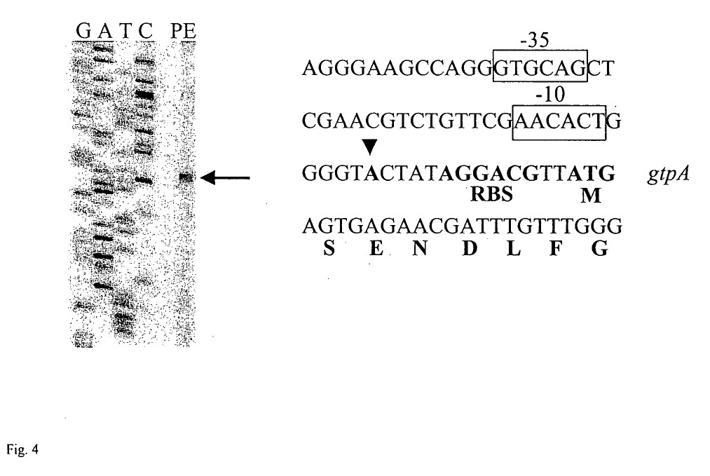

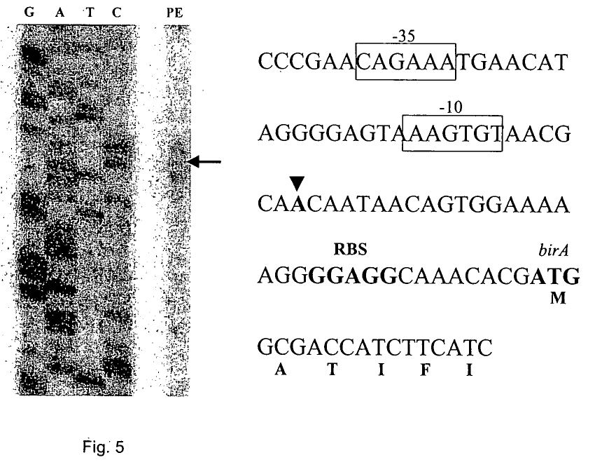

If the vegetative B. infantis RNA polymerase recognises promoter sequences similar to those from other bacteria (i.e. −10: TATAAT and −35 being TTGACA), putative promoter motifs (FIGS. 4 and 5) may be proposed upon inspection of the DNA sequence immediately upstream of the TSS. As yet no definitive consensus sequence can be determined from these motifs, which may be due to the fact that these RNA polymerase recognition sites can tolerate a significant amount of degeneracy, or that the sequences examined are not representative of typical bifidobacterial −10 and −35 hexamers. It is also possible that the recognition sites of the vegetative RNA polymerase in Bifidobacterium are dissimilar to those previously reported for a variety of bacterial species.

Throughout the specification the term derivative is taken to include active forms of the protein with modifications which do not substantially effect the activity of the protein. The term mutant is taken to include amino acid variations which do not substantially effect the activity of the protein. Sequence mutants have a greater than 96% identity with the parent DNA sequence. The term fragment is taken to include units encoded by a nucleic acid sequence present in all or part of the amino acid sequences corresponding to all or part of the nucleic acid sequences disclosed herein. In this context the term part means at least 10, preferably at least 15, preferably at least 20 amino acids.

The invention will be more fully understood from the following examples.

### Materials and Methods

Bacterial Strains, Media, Chemicals and Culture Conditions

Strains and plasmids used in this study are listed in Table 1 below. Bifidobacteria were routinely cultured in de Man, Rogosa and Sharpe medium (MRS (12); Oxoid Ltd., Hampshire, England) supplemented with 0.2% (w/v) glucose. MRS was supplemented with 0.05% (w/v) cysteine-HCl, and strains were grown at 37° C. under anaerobic conditions maintained using the Anaerocult oxygen depleting system (Merck, Darmstadt, Germany) in an anaerobic chamber. Escherichia coli strains were grown in Luria-Bertani (LB) medium at 37° C. with agitation (46). Stocks of all cultures were maintained at −20° C. in 40% glycerol. When necessary antibiotics were added to the media as follows: ampicillin (100 μg ml⁻¹ (50 μg ml⁻¹ in the case of plasmid pWSK29)), tetracycline (12.5 μg ml⁻¹), or chloramphenicol (20 μg ml⁻¹). X-Gal and 5-bromo-4-chloro-3-indolyl phosphate (X-P) were used at final concentrations of 40 μg ml⁻¹.

TABLE 1
Bacterial strains and plasmids

| Bacterial strain | Relevant | Source or |
|---|---|---|
| plasmid | properties | reference |
| Strains | | |
| E. coli | recAl, endAl, gyrA96, | Stratagene Ltd., |
| XL1-Blue | thi-1, hsdR17, supE44, relAl, | Cambridg UK. |
| | lac [F′ proAB lact^{q}ZΔ | |
| | M15 Tn10(Tc^{r})] | |
| E. coli | PhoR and CreC mutations | (31) |
| ANCC22 | | |
| E. coli | PhoB ⁻ | (31) |
| ANCL1 | | |
| E. coli | ΔnarQ251::Tn10d | (42) |
| VJS3051 | (Tc^{r})ΔnarX242 zch- | |
| | 2084::Ω-Cm^{r}Φ(fdnG-lacZ) | |
| E. coli | Δ(lac-argF)U169 λΦ(fdnG-lacZ) | (43) |
| VJ53081 | narL215::Tn10 | |
| Bifidobacterium | Wild-type human isolate | UCC Culture |
| infantis | | Collection |
| UCC35624 | | |
| Plasmids | | |
| pBluescript KS⁻ | Ap^{r} αlacZ | Stratagene Ltd. |
| pWSK29 | Ap^{r} αlacZ, low copy number | (59) |

DNA Manipulations and Sequence Analysis

Plasmid DNA was obtained from E. coli by using either an alkaline lysis method (8) or the QIAprep Spin Plasmid Miniprep kit (Qiagen GmbH, Hilden, Germany). Large scale preparation of total DNA from B. infantis was prepared as described previously (34). Purified DNA was obtained by caesium chloride ultracentrifugation of this preparation, as described by Sambrook et al. (46). Restriction endonucleases, T4 DNA ligase and calf intestinal alkaline phosphatase were purchased from Roche Diagnostics Ltd. (Lewes, East Sussex, UK) or New England Biolabs Ltd. (Hitchin, UK), and used as recommended by the manufacturers. Electroporation of plasmid DNA into E. coli was performed essentially as previously described (46). PCR reactions were accomplished using either the Taq PCR Master Mix (Qiagen, as above) or the Expand Long Template PCR System (Roche Diagnostics GmbH, Mannheim, Germany) in accordance with the manufacturer's instructions. PCR reactions were executed using an Omnigene thermal cycler (Hybaid Ltd., Middlesex, UK). Sequencing was performed by MWG-BIOTECH AG (Ebersberg, Germany). Sequence data assembly and analysis were performed using DNASTAR software (DNASTAR, Madison, Wis., USA). Database searches were performed using non-redundant sequences at the NCBI internet site (http://www.ncbi.nlm.nih.gov) using tBlastN, tBlastX and BlastP programs (2, 3). Sequence alignments were performed using the Clustal Method of the MEGALIGN program of the DNASTAR software package. Functional domains in deduced proteins were identified using the SMART database (48, 49) internet site (http://smart.embl-heidelberg.de).

Phenotypic Complementation and Activity Assays of Mutant Strains

Ligation mixes were prepared essentially as described previously (32). The ligation mixes were introduced into competent E. coli ANCC22 or VJS3051 by electrotransformation (46) using the Bio-Rad Gene Pulser apparatus according to the manufacturer's instructions (Bio-Rad Laboratories, Richmond, Calif. USA). Colonies phenotypically exhibiting increased activity (alkaline phosphatase (AP) activity on XP plates in the case of strain ANCC22, or P-gal activity on X-gal plates for strain VJS3051), as indicated by the formation of a blue-coloured colony, were selected for quantitative assay. AP activity assays were performed as described previously (1).

Degenerate PCR

PCR was performed on B. infantis UCC35624 chromosomal DNA, using degenerate oligonucleotide primers designed specifically to correspond to conserved regions of RRs, essentially as previously described (30). Sequences of (assumed) RRs from bacteria with high G+C %-content were obtained from the BLAST database and aligned using the MEGALIGN program from DNASTAR. Conserved residues were identified (approximately 97 amino acids apart) and degenerate primers (MWG-BIOTECH, Ebersburg, Germany) were designed on these. Two different forward oligonucleotides, GT(G/A/T/C)GT(G/A/T/C)GA(G/A/T/C)GA(C/T)GA and A/C)T(G/A/T/C)GT(G/A/T/C)GA(G/A/T/C)GA(C/T)GA, corresponding to the amino acids VV(DE)D(DE) and (ILM)V(DE)D(DE), respectively; and one reverse oligonucleotide, (A/G)(A/T)A(A/G)TC(G/A/T/C)GC(G/A/T/C)CC, corresponding to the amino acid sequence GAD(IN), were designed based on conserved amino acid residues around the DD and K boxes of known RRs (30). PCR conditions were essentially as previously described (30). Fragments of the expected size (approximately 300 bp) were excised from 2% agarose gels, purified using the CONCERT™ Rapid PCR Purification system (GibcoBRL, Paisley, Scotland) and cloned into pCR®2.1-TOPO® vector prior to sequencing.

Anchored PCR and Southern Hybridisation

Anchored PCR was used in order to obtain the DNA sequence surrounding the cloned ORF specifying the assumed HPK or RR, essentially as previously described (11). PCR products were purified and used for sequencing purposes. Restricted chromosomal DNA from B. infantis UCC 35624 was separated by agarose gel electrophoresis and transferred to nylon membranes (Hybond N⁺, Amersham International, Little Chalfont, Bucks, UK) by the method of Southern (50) as modified by Wahl et al. (58). DNA was labelled using the Enhanced Chemiluminescence (ECl) gene detection system (Amersham, as above). Probe labelling, hybridisation conditions and washing steps were completed according to the manufacturer's instructions.

RNA Isolation, Northern Analysis and 5′ Extension Analysis

Northern analysis was performed on aliquots of total RNA extracted using the Macaloid method (21) from bifidobacterial cultures which had been harvested at a range of optical density at 600 nm (O.D. 600) values between 0.2 and 1.4. RNA samples were treated with DNase and RNase inhibitor (Roche Diagnostics), denatured at 70° C. for 10 min, and loaded with formamide-containing dye on to a 1.2% formaldehyde gel (6). RNA size standards from Promega (Madison, Wis., USA) were used to enable transcript size estimation. Capillary blotting to Hybond-N+ nylon membranes (Amersham, as above) was performed essentially as previously described (46). An internal 500 bp fragment (amplified using PCR) from each ORF identified for each of the three 2CS-encoding loci was used as a probe (for primer sequences see Table 2 below). The probes were radiolabelled with γ-³²P using a Prime-a-Gene kit (Promega, as above).

TABLE 2

Primers utilised to amplify the internal
fragments of genes described in this study to
be used as probes for Northern hybridisations

| Gene | Forward primer | Reverse primer |
|---|---|---|
| gtpA | GCAACAGTCTCACGATTC | GGGGCGTTCCTCAAATAC |
| birA | AACACCATGGCGACCATC | TCCATCGGAGTGAGATTC |
| bikA | AGTCTGATTTCTGACGAC | GTGGTCACCGGGGTACGC |
| lipA | TGGGTTCCTTGGATTCGC | CACATTTGCGTCGGCATC |
| biaA | GATTGGTGCCAAGAAGGC | CGGGGTGCGTGGCCAGCC |
| biaB | GCCAAGGTCATCACCTCC | GCCTGCATCACGCAGATC |
| biaC | TTCGGCCTGCTGGCCGGC | GGAGCCGAGCACGTAGCC |
| birB | GACGTCATGCTGCCTGAC | GGTCACGTCGTGGGAGTC |
| bikB | GCCGAATTCAGCCTTGCC | GGACTGCTTGGGCTCAGG |
| bikC | TCGAGCACATGGTCGGCC | CTGCGCCAGCGTCCAGGC |
| birC | CGTGAGGGGCTGCGCGCC | TTGTGTGCGGTCGGCGAC |
| bicC (3′) | CTGCTGGCCGAAGCGGCG | GGCGCACCAGTTCGACGC |
| bicC (5′) | GAGATCCACAGCACCAGC | GAATTCAAGGACGATTAC |

Primer extension (PE) to identify the transcriptional start site (TSS) was accomplished by annealing γ-³³P-radiolabelled synthetic oligonucleotides to RNA as previously described (41). Primers were designed approximately 100 bp downstream of the predicted ribosome binding site (RBS) of the assumed first coding sequence of each transcript, and PE was performed by annealing 5 pmol γ-³³P-labelled primer to 50 μg of RNA. Sequence ladders for each of the PE reactions were produced, using the same primer as used for the PE, and with the aid of the T7 DNA Polymerase Sequencing Kit (USB Corp., Ohio, USA). The Genbank accession numbers for the three regions specifying 2CSs identified are as follows: System A, AY266333; System B, AY266334; System C, AY266335.

Functional Complementation of E. coli ANCC22

Using a complementation strategy as described above, fifteen transformants, each carrying (a) random chromosomal fragment(s) of B. infantis UCC 35624 cloned into the high copy number pBluescript vector, were shown to be capable of suppressing the E. coli ANCC22 PhoA-negative phenotype on solid media. This phenotypic suppression strategy was also employed without success using a second mutant E. coli strain, VJS3051 (42), and a low copy number vector (pWSK29) (data not shown). The complementing ANCC22 clones were quantitatively assayed for increased AP activity. All transformants exhibited increased AP activity, ranging from 40 to 200 units as compared to a negative control of ANCC22 containing pBluescript (<5 units). Furthermore, introduction of the recombinant plasmids from the suppressed isolates into the control strain ANCL1 showed that suppression was not due to the cloning of a phosphatase, or a regulator of phoA transcription, as outlined previously (31). Sequence data for the inserts (ranging from 1 to 2 kb) of each plasmid capable of phenotypic suppression revealed the presence of (varying 3′ sections of) a single HPK-encoding gene, corresponding to the transmitter domain of this assumed HPK (designated bikA, see below).

Identification of Two Putative RR-Encoding Genes Using Degenerate PCR

Sequence comparison of 50 independent plasmid inserts obtained using PCR allowed the identification of two ORFs, each displaying significant similarity with the N-terminal internal fragment of a RR-encoding gene. These assumed RR-encoding genes were designated birB and birC (Table 3 below). The PCR product encoding BirB was obtained using the forward primer VV(DE)D(DE) in conjunction with the reverse primer (see above). The second RR-encoding moiety, birC, was obtained using the second degenerate primer, (ILM)V(DE)D(DE), with the reverse primer.

TABLE 3
Classification and putative functional domains of HPKs and RRs identified

| | | N-terminal | | | C-terminal | Class/ |
|---|---|---|---|---|---|---|
| ORF | Size (aa) | domains (aa) | HAMP^{a} | HPKA^{b} | domains (aa) | Group |
| HPK | | Transmembrane | | | HATPase-c^{c} | Group |
| BikA | 565 | 29-51 | 210-278 | 290-356 | 402-513 | IIIA |
| | | 172-194 | | | | |
| | | 207-229 | | | | |
| BikB | 448 | 51-73 | 69-121 | 134-202 | 266-413 | IIIA |
| BikC | 348 | N/A | N/A | 172-241 | 275-321 | II |
| | | | | | Effector | |
| RR | | Receiver domain | | | domain | |
| | | (homology) | | | (homology) | |
| BirA | 240 | CheY | | | PhoP/OmpR | 2 |
| BirB | 227 | CheY | | | OmpR/PhoB | 2 |
| BirC | 214 | CheY | | | NarL/DegU | 3 |

^{a}HAMP: Histidine kinase, adenlyl cyclase, methyl binding protein, phosphatase domain
^{b}HPK-A: Histidine kinase A motif
^{c}HATPase-c: Histidine kinase-, DNA gyraseB-, phytochrome-like ATPase
N/A: not apparent

Comparative Sequence Analysis of the Three 2CSs.

Analysis of the DNA regions surrounding bikA, birB and birC, which were obtained by anchored PCR, showed that each gene was flanked by either an RR- or a HPK-encoding gene, thus revealing three complete 2CSs. Additional ORFs were identified in some cases.

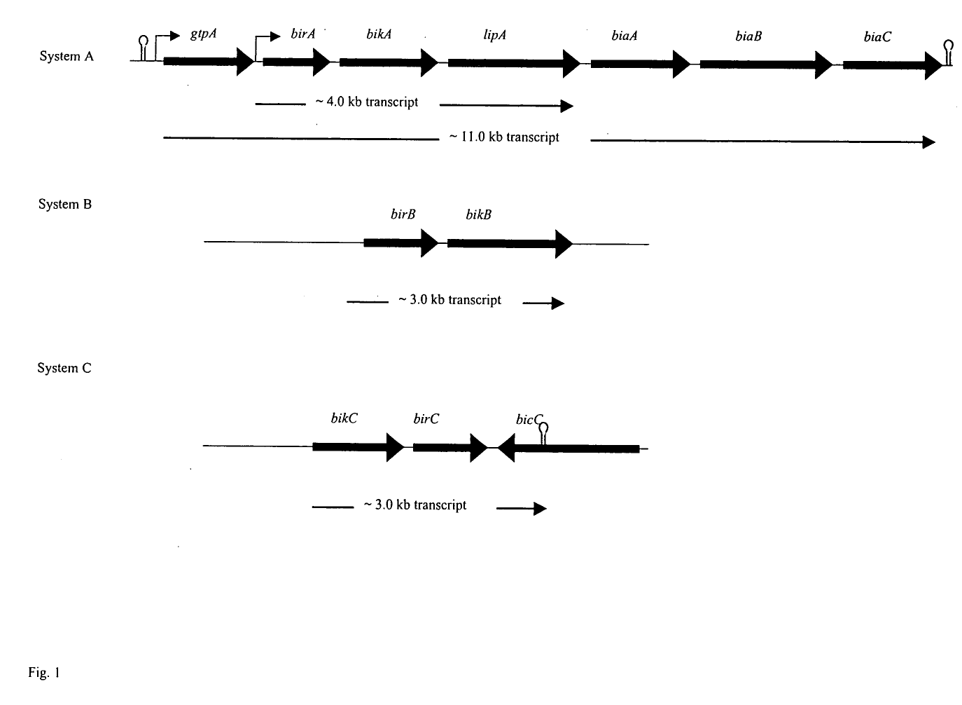

All identified ORFs are schematically depicted in FIG. 1 and summarised in Table 4 along with a number of their salient features. bikA is located immediately downstream of its cognate RR-encoding gene, birA (birA-bikA was designated System A). This genetic organisation was also observed for birB-bikB (referred to as System B). In contrast, bikC is located immediately upstream of its cognate RR-encoding gene, birC (bikC-birC was named System C). HAMP domains (cytoplasmic helical linker domains proposed to have a role in the regulation of the phosphorylation of the HPK and present in many prokaryotic signalling proteins (4)), HPK-A motifs (the predicted dimerisation and phosphoacceptor domain) and HATPase-c domains (histidine kinase-like ATPase; involved in ATP-binding) were identified in each of these two HPKs (Table 3) using the SMART database.

A 1380 bp ORF is located immediately upstream of birA, and the deduced protein product of this gene, designated gtpA, displays high similarity to a GTP-binding protein. A predicted lipoprotein-encoding ORF, designated lipA, was identified downstream of the HPK. Downstream of lipA, three genes were identified which appear to constitute a putative ABC transport system. The gene organisation of the System A operon (Table 4) is conserved in B. longum DJO10A, B. longum NCC2705 and B. breve NCIMB 8807 (FIG. 2). A partly homologous gene cluster consisting of the first four genes of this operon is found in a number of Mycobacterium spp. (FIG. 2). Interestingly, while clear homologues were found for Systems A and B in other sequenced Bifidobacterium spp., this was not the case for System C, although the bicC gene (located immediately downstream of System C) was clearly present in B. longum (Table 4).

A number of putative Rho-independent transcriptional terminator structures were identified on the basis of being able to form stable stem-loop structures (ΔG<-15 kcal mol⁻¹) and are depicted in FIG. 1. No putative hairpin structures with significant ΔG values were identified immediately downstream of lipA; however, a region rich in C and poor in G was detected (13% G over 60 bases), suggesting the involvement of a rho-dependent terminator (24).

TABLE 4
2CSs identified in B. infantis UCC35624 and
their surrounding ORFs (see also FIG. 2)

| | | ORF with the | | | |
|---|---|---|---|---|---|
| | Size | highest similarity | | | |
| ORF | (aa) | score | Organism | Identity (%) | P value |
| System A | | | | | |
| GtpA | 459 | Blon_22 | B. longum DJO10A | 96 | 0.0 |
| BirA | 240 | Blon_22 | B. longum DJO10A | 80 | 1e−101 |
| BikA | 565 | Blon_22 | B. longum DJO10A | 96 | 0.0 |
| LipA | 467 | hypothetical | B. longum NCC2705 | 93 | 0.0 |
| | | protein | | | |
| BiaA | 324 | Blon_22 | B. longum DJO10A | 93 | 1e−167 |
| BiaB | 504 | ATP-binding | B. longum NCC2705 | 100 | 0.0 |
| | | protein of ABC | | | |
| | | transporter | | | |
| BiaC | 696 | probable ABC | B. longum NCC2705 | 89 | 0.0 |
| | | transport system | | | |
| | | permease protein | | | |
| System B | | | | | |
| BirB | 227 | Blon_18 | B. longum DJO10A | 91 | 2e−93 |
| BikB | 448 | Blon_18 | B. longum DJO10A | 96 | 0.0 |
| System C | | | | | |
| BikC | 348 | hypothetical | P. syringae pv. | 39 | 2e−19 |
| | | protein | syringae B728a | | |
| BirC | 214 | segment 14/29 | S. coelicolor A3(2) | 40 | 1e−21 |
| BicC | 613 | Blon_30 | B. longum DJO10A | 92 | 0.0 |

Transcriptional Regulation and 5′ Extension Analysis

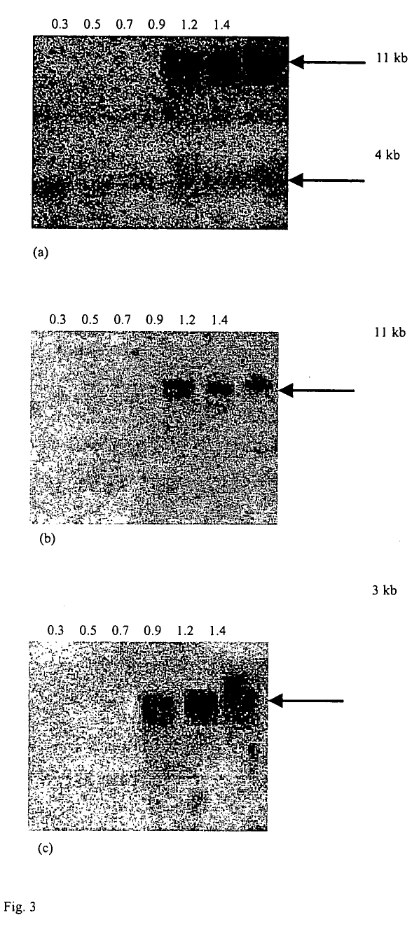

Northern analysis was performed to elucidate the manner in which the three 2CSs-encoding loci are transcribed. All probes used for System A hybridised to a large (11 kb) transcript, indicating that all these genes are co-transcribed, thus comprising an 11 kb-long operon (FIGS. 3a and 3b). In addition, a smaller transcript of 4 kb was observed only when the probes derived from birA, bikA and lipA were used (FIG. 3b). This second transcript was constitutively expressed from early exponential to late stationary phase. On the other hand, the 11 kb transcript was evident only from late exponential to late stationary phase. Both gene probes obtained from bikB and birB in System B hybridised to a transcript of 3.0 kb, in mRNA samples obtained from cells at late exponential- to late stationary-growth phase, indicating that these genes are transiently transcribed as a dicistronic operon (FIG. 3c). Similarly, bikC- and birC-derived probes hybridised to a single 3.0 kb mRNA transcript only from mRNA of late exponential- to late stationary-phase cells. A probe obtained from bicC, encompassing DNA on the 5′ side of the putative transcriptional terminator (FIG. 1) also hybridised to a similar sized transcript, whereas a bicC probe consisting of DNA located at the 3′ side of this stem-loop structure did not (results not shown).

Primer extension analysis was attempted for each system to elucidate the transcription start site (TSS) of the four identified transcripts. The TSS for the large 11 kb transcript of System A was identified as an adenine base, situated 13 bp upstream of the assumed start codon of gtpA (FIG. 4). The TSS for the putative promoter immediately proximal to birA was identified as an adenine residue, situated 33 bp upstream of the presumed translational start site of birA, as indicated in FIG. 5. No definitive sequence ladder-primer extension pair could be obtained for either of Systems B or C despite exhaustive attempts.

Without wishing to be bound by theory, it is believed that the proteins and DNA sequences encoding the proteins for the ABC transporter system proteins A (SEQID10) and C (SEQID11) of system A may be useful in managing and altering the transport of nutrients, metabolites, proteins and other biological molecules into and out of the bacterial cell. Such transport management and alteration may enable the optimisation of growth conditions to obtain a growth end-point in the cell (such as, for example, bacteriostasis, or sporulation) by enabling the identification of key nutrients or metabolites transported to or from the cell. Furthermore, the proteins, and the genes encoding them may allow for the genetic modification of other unrelated bacterial strains, so as to allow for the transport of those nutrients, and subsequent initiation of the growth endpoint referred to above.

The 2CS proteins and sequences of the invention relate to a sensing system of the bacteria. These systems usually carry out environmental sensing, such as pH, nutrient concentration, temperature and the like. They are important for enabling the correct expression of required proteins to maintain bacterial viability. The 2CS systems operate by causing phosphorylation and dephosphorylation of effector proteins, which in turn are activated or deactivated—leading to signal transduction cascades that eventually result in the activation or suppression of certain systems. The 2CS systems isolated from Bifidobacterium infantis UCC 35624 are important in enabling its probiotic activity, as it is their environmental sensing that switches on the appropriate systems in the gut. Therefore, it is possible that they may be responsible, and certainly likely that they are at least involved, in the probiotic activity of UCC 35624; by enabling the correct array of gene expression.

The proteins/sequences of the invention may be cloned into non-probiotic bacteria to enable them to become probiotic by adjusting gene expression in the gastrointestinal tract. They also can be used to screen for potentially probiotic bacteria. Bacteria can be tested to determine if they have such two component systems, and are the 2CS systems modified by pH and other environmental parameters. In particular, the C system appears to be unique to Bifidobacteria and can therefore be used to screen samples for the presence of Bifidobacteria.

The invention is not limited to the embodiments hereinbefore described which may be varied in detail.

References

1. Aiba, H., M. Nagaya, and T. Mizuno. 1993. Sensor and regulator proteins from the cyanobacterium Synechococcus species PCC7942 that belong to the bacterial signal-transduction protein families: implication in the adaptive response to phosphate limitation. Mol. Microbiol. 8:81-91

2. Altschul, S. F., W. Gish, W. Miller, E. W. Myers, and D. J. Lipman. 1990. Basic local alignment search tools. J. Mol. Biol. 215:403-410.

3. Altschul, S. F., T. L. Madden, A. A. Schafer, J. Hang, Z. Hang, W. Miller, and D. J. Lipman. 1997. Gapped Blast and PSI-Blast: a new generation of protein database search programs. Nucleic Acids Res. 25:3389-3402.

4. Aravind, L. and C. P. Ponting. 1999. The cytoplasmic helical linker domain of receptor histidine kinase and methyl-accepting proteins is common to many prokaryotic signalling proteins. FEMS Microbiol Lett. 176:111-116.

5. Arthur M., F. Depardieu, and P. Courvalin. 1999. Regulated interactions between partner and non-partner sensors and response regulators that control glycopeptide resistance gene expression in enterococci. Microbiology 145:1849-1858.

6. Ausubel, F. M., R. Brent, R. E. Kingston, D. D. Moore, J. A. Seidman, J. A. Smith, and K. Struhl. 1987. Current protocols in molecular biology. Greene Publishing Associates and Wiley-Interscience, New York, N.Y.

7. Baikalov, I., I. Schroder, M. Kaczor-Grzeskowiak, K. Grzeskowiak, R. P. Gunsalus and R. E. Dickerson. 1996. Structure of the Escherichia coli response regulator NarL. Biochemistry 35:11053-11061.

8. Birnboim H. C., and J. Doly. 1979. A rapid alkaline extraction procedure for screening recombinant plasmid DNA. Nucleic Acids Res. 7:1513-1523.

9. Brock T. D., 1997. Chapter 16. Prokaryotic Diversity: Bacteria. p. 635-740. In M. T. Madigan, J. M. Martinko and J. Parker (ed.), Biology of Microorganisms, 8th ed. Prentice-Hall, Inc., New Jersey.

10. Chang, C., and E. M. Meyerowitz. 1995. The ethylene hormone response in Arabidopsis: a eukaryotic two-component signalling system. Proc. Natl. Acad. Sci. USA. 92: 4129-4133.

11. Cintas, L. M., P. Casaus, H. Holo, P. E. Hernandez, I. F. Nes, and L. S. Håvarstein. 1998. Enterocins L50A and L50B, two novel bacteriocins from Enterococcus faecium L50, are related to staphylococcal hemolysins. J. Bacteriol. 180:1988-1994.

12. de Man, J., M. Rogosa, and M. E. Sharpe. 1960. A medium for the culture of lactobacilli. J. Appl. Bacteriol. 23:130-135.

13. Dunne, C. 2001. Adaptation of bacteria to the intestinal niche: probiotics and gut disorder. Inflamm. Bowel Dis. 7:136-145.

14. Dunne, C., L. Murphy, S. Flynn, L. O'Mahony, S. O'Halloran, M. Feeney, D. Morrissey, G. Thornton, G. Fitzgerald, C. Daly, B. Kiely, E. M. Quigley, G. C. O'Sullivan, F. Shanahan, and J. K. Collins. 1999. Probiotics: from myth to reality. Demonstration of functionality in animal models of disease and in human clinical trials. Antonie Van Leeuwenhoek. 76:279-292.

15. Fabret, C., V. A. Feher, and J. A. Hoch. 1999. Two-component signal transduction in Bacillus subtilis: how one organism sees its world. J. Bacteriol. 181:1975-1983.

16. Fleischmann R. D., M. D. Adams, O. White, R. A. Clayton, E. F. Kirkness, A. R. Kerlavage, C. J. Bult, J. F. Tomb, B. A. Dougherty, J. M. Merrick, K. McKenney, G. G. Sutton, W. FitzHugh, C. A. Fields, J. D. Gocayne, J. D. Scott, R. Shirley, L. I. Liu, A. Glodek, J. M. Kelley, J. F. Weidman, C. A. Phillips, T. Spriggs, E. Hedblom, M. D. Cotton, T. Utterback, M. C. Hanna, D. T. Nguyen, D. M. Saudek, R. C. Brandon, L. D. Fine, J. L. Fritchman, J. L. Fuhrmann, N. S. Geoghagen, C. L. Gnehm, L. A. McDonald, K. V. Small, C. M. Fraser, H. O. Smith, and C. J. Venter 1995. Whole-genome random sequencing and assembly of Haemophilus influenzae Rd. Science 269:496-512.

17. Fuller R. 1989 Probiotics in man and animals. J Appl Bacteriol. 66:365-378.

18. Guarner F., and G. J. Schaafsma. 1998 Probiotics. Int J Food Microbiol. 39:237-238.

19. Hung, M.-N., Z. Xia, N.-T. Hu, and B. H. Lee. 2001. Molecular and biochemical analysis of two β-galactosidases from Bifidobacterium longum HL96. Appl. Env. Microbiol. 67:4256-4263.

20. Kim, D.-J., and S. Forst. 2001. Genomic analysis of the histidine kinase family in bacteria and archaea. Microbiol. 147:1197-1212.

21. Kuipers, O. P., M. M. Beerthuyzen, R. J. Siezen, and W. M. de Vos. 1993. Characterisation of the Nisin gene cluster nisABTCIPR of Lactococcus lactis, requirement of expression of the nisA and nisI genes for development of immunity. Eur. J. Biochem. 216:281-291.

22. Lee, P. J., and A. M. Stock. 1996. Characterisation of the genes and proteins of a two-component system from the hyperthermophilic bacterium Thermotoga maritima. J. Bacteriol. 178:5579-5585.

23. Lee, Y. K., and S. Salminen. 1995. The coming age of probiotics. Trends Food Sci. Technol. 6:241-245.

24. Lewin, B. 1995. Part 4: Control of prokaryotic gene expression, p. 375-523. In Genes V. Oxford University Press, Oxford, U.K.

25. Loomis, W. F., G. Shaulsky, and N. Wang. 1997. Histidine kinases in signal transduction pathways of eukaryotes. J. Cell Science. 110:1141-1145.

26. Matsumura, H., Takeuchi, A., and Y. Kano. 1997. Construction of an Escherichia coli-Bifidobacterium longum shuttle vector transforming B. longum 105-A and 108-A. Biosci. Biotechnol. Biochem. 61:1211-1212.

27. Minowa, T., S. Iwata, H. Sakai, H. Masaki, and T. Otha. 1989. Sequence and characteristics of the Bifidobacterium longum gene encoding L-lactate dehydrogenase and the primary structure of the enzyme: a new feature of the allosteric site. Gene. 85:161-168.

28. Mizuno, T. 1997. Compilation of all genes encoding two-component phosphotransfer signal transducers in the genome of E. coli. DNA Research. 4:161-168

29. Morel-Deville, F., F. Fauvel, and P. Morel. 1998. Two-component signal-transducting systems involved in stress response and vancomycin susceptibility in Lactobacillus sakei. Microbiol. 144:2873-2883.

30. Morel-Deville, F., S. D. Ehrlich, and P. Morel. 1997. Identification by PCR of genes encoding multiple response regulators. Microbiol. 143:1513-1520.

31. Nagasawa, S., K. Ishige, and T. Mizuno. 1993. Novel members of the two-component signal transduction genes in E. coli. J. Biochem. 114:350-357.

32. O'Connell-Motherway, M., G. F. Fitzgerald, and D. van Sinderen. 1997. Cloning and sequence analysis of putative histidine protein kinases isolated from Lactococcus lactis MG1363. Appl. Environ. Microbiol. 63:2454-2459.

33. O'Connell-Motherway, M., D. van Sinderen, F. Morel-Deville, G. F. Fitzgerald, S. Dusko Ehrlich, and P. Morel. 2000. Six putative two-component regulatory systems isolated from Lactococcus lactis subsp. cremoris MG1363. Microbiology. 146:935-947.

34. O'Riordan, K. 1998. Studies on antimicrobial activity and genetic diversity of Bifidobacterium species: molecular characterisation of a 5.75 kb plasmid and a chromosomally encoded recA gene homologue from Bifidobacterium breve. Ph.D. thesis, Department of Microbiology, National University of Ireland, Cork, Ireland.

35. Pao, G. M., and M. H. Saier (Jr). 1995. Response regulators in bacterial transduction systems: selective domain shuffling during evolution. J. Mol. Evol. 40:136-154.

36. Pao, G. M. and M. H. Saier (Jr). 1997. Nonplastid eucaryotic response regulators have a monophyletic origin and evolved from their bacterial precursors in parallel with their cognate sensor kinases. J. Mol. Evol. 44:605-613.

37. Parkinson, J. S., and E. C. Kofoid. 1992. Communication modules in bacterial signalling proteins. Ann. Rev. Genet. 26:71-112.

38. Parkinson, J. S. 1993. Signal transduction schemes in bacteria. Cell. 73:857-871.

39. Parkinson, J. S. 1995. Genetic approaches for signalling pathways and proteins. In Two-Component Signal Transduction. J Hoch and T. Silhavy (eds).

40. Phalip, V., J. H. Li, and C. C. Zhang. 2001. HstK, a cyanobacterial protein with both a serine/threonine kinase domain and a histidine kinase domain: implication for the mechanism of signal transduction. Biochem J. 360:639-644.

41. Pujic, P., R. Dervyn, A. Sorokin, and S. D. Ehrlich. 1998. The kdgRKAT operon of Bacillus subtilis: detection of the transcript and regulation by the kdgR and ccpA genes. Microbiology 144: 3111-3118.

42. Rabin, R. S., and V. Stewart. 1992. Either of two functionally redundant sensor proteins, NarX and NarQ, is sufficient for nitrate regulation in Escherichia coli K-12. Proc. Natl. Acad. Sci. 89:8419-8423.

43. Rabin, R. S., and V. Stewart. 1993. Dual response regulators (NarL and NarP) interact with dual sensors (NarX and NarQ) to control nitrate- and nitrite-regulated gene expression in Escherichia coli K-12. J. Bacteriol. 175:3259-3268.

44. Rossi, M., L. Altomare, A. Gonzalez Vara y Rodriguez, P. Bridigi, and D. Matteuzzi. 2000. Nucleotide sequence, expression and transcriptional analysis of the Bifidobacterium longum MB 219 lacZ gene. Arch. Microbiol. 174:74-80.

45. Rossi, M., P. Bridigi, and D. Matteuzzi. 1997. An efficient transformation system for Bifidobacterium spp. Lett. Appl. Microbiol. 24:33-36.

46. Sambrook, J., E. F. Fritsch, and T. Maniatis. 1989. Molecular cloning. A laboratory manual. 2nd edn, Cold Spring Harbor, N.Y.: Cold Spring Harbor Laboratory.

47. Schell, M. A., M. Karmirantzou, B. Snel, D. Vilanova, B. Berger, G. Pessi, M.-C. Zwahlen, F. Desiere, P. Bork, M. Delley, R. D. Pridmore, and F. Arigoni. 2002. The genome sequence of Bifidobacterium longum reflects its adaptation the human gastrointestinal tract. Proc. Natl. Acad. Sci. 99:14422-14427.

48. Schultz, J., R. R. Copley, T. Doerks, C. P. Ponting, and P. Bork. 2000. SMART: a web based tool for the study of genetically mobile domains. Nucleic Acids Res. 28:231-234.

49. Schultz, J., F. Milpetz, P. Bork, and C. P. Ponting. 1998. SMART, a simple modular architecture research tool: identification of signalling domains. Proc. Natl. Acad. Sci. USA. 95:5857-5864.

50. Southern, E. M. 1975. Detection of specific sequences among DNA fragments separated by gel electrophoresis. J. Mol. Biol. 98:503-517.

51. Stanton, C., G. Gardiner, H. Meehan, K. Collins, G. Fitzgerald, P. B. Lynch, and R. P. Ross. 2001. Market potential for probiotics. Am. J. Clin. Nutr. 73:476S-483S.

52. Stewart, V., J. Parales, Jnr., and S. M. Merkel. 1989. Structure of genes narL and narX of the nar (nitrate reductase) locus in Escherichia coli K-12. J. Bacteriol. 171:2229-2234.

53. Stock, J. B., M. G. Surette, M. Levit, and P. Park. 1995. Two-component signal transduction systems: structure-function relationships and mechanisms of catalysis. In Two-Component Signal Transduction. J. A. Hock and T. J Silhavy (eds). ASM Press, Washington DC.

54. Tannock, G. W. 1999. Probiotics: a critical review. Horizon Scientific Press, England.

55. Tannock, G. W. 1997. Probiotic properties of lactic-acid bacteria: plenty of scope for fundamental R & D. Trends Biotechnol. 15:270-274.

56. Utusumi, R., S. Katayama, M. Taniguchi, T. Horie, M. Ikeda, S. Igaki, H. Nakagawa, A. Miwa, H. Tanabe, and M. Noda. 1994. Newly identified genes involved in the signal transduction of E. coli K-12. Gene 140:73-77.

57. Volz, K. 1995. Structural and functional conservation in response regulators. In Two-Component Signal Transduction. J. A. Hock and T. J Silhavy (eds). ASM Press, Washington DC.

58. Wahl, G., M. Stern, and G. R. Stark. 1979. Efficient transfer of large DNA fragments from agarose gels to diazobenzylomethyl paper and rapid hybridisation using dextran sulphate. Proc. Natl. Acad. Sci. USA. 76:3683-3687.

59. Wang, R. F., and S. R. Kushner. 1991. Construction of versatile low-copy-number vectors for cloning, sequencing and gene expression in Escherichia coli. Gene. 100:195-199.

1: An isolated Bifidobacteria DNA fragment comprising nucleic acid sequence ID No. 1 or a mutant or fragment or variant thereof.

2: An isolated Bifidobacteria DNA fragment comprising nucleic acid sequence ID No. 2 or a mutant or fragment or variant thereof.

3: An isolated Bifidobacteria DNA fragment comprising nucleic acid sequence ID No. 3 or a mutant or fragment or variant thereof.

4: A DNA fragment comprising nucleic acid sequence ID No. 10 or a mutant or fragment or variant thereof.

5: A protein encoded by the DNA fragment of claim 4 or a mutant or fragment or variant thereof.

6: A DNA fragment comprising nucleic acid sequence ID No. 11 or a mutant or fragment or variant thereof.

7: A protein encoded by the DNA fragment of claim 6 or a mutant or fragment or variant thereof.

8: A protein having sequence ID No. 4 or a mutant or fragment or variant thereof.

9: A protein having sequence ID No. 5 or a mutant or fragment or variant thereof.

10: A protein having sequence ID No. 6 or a mutant or fragment or variant thereof.

11: A protein having sequence ID No. 7 or a mutant or fragment or variant thereof.

12: A protein having sequence ID No. 8 or a mutant or fragment or variant thereof.

13: A protein having sequence ID No. 9 or a mutant or fragment or variant thereof.

14: A DNA fragment or protein as claimed in claim 1 isolated from the probiotic genus Bifidobacterium.

15: A DNA fragment or protein as claimed in claim 1 isolated from Bifidobacterium infantis UCC35624.

16: A two-component signal transduction system comprising a gene encoding sequence ID No. 4 or a mutant or fragment or variant thereof and a gene encoding sequence ID No. 5 or a mutant or fragment or variant thereof.

17: A two-component signal transduction system comprising a gene encoding sequence ID No. 6 or a mutant or fragment or variant thereof and a gene encoding sequence ID No. 7 or a mutant or fragment or variant thereof.

18: A two-component signal transduction system comprising a gene encoding sequence ID No. 8 or a mutant or fragment or variant thereof and a gene encoding sequence ID No. 9 or a mutant or fragment or variant thereof.

19: A two-component signal transduction system as claimed in claim 16 isolated from the probiotic genus Bifidobacterium.

20: A two-component signal transduction system as claimed in claim 16 isolated from Bifidobacterium infantis UCC35624.

21: A protein encoded by a DNA fragment comprising sequence ID No. 1, sequence ID No. 2 or sequence ID No. 3 or a derivative, fragment or mutant thereof.

22 : A method of screening for the presence of Bifidobacteria using a DNA fragment comprising sequence ID No. 1, sequence ID No. 2 or sequence ID No. 3 or sequence ID No. 10 or sequence ID No. 11 or a derivative, fragment or mutant thereof.

23 : A method of screening for the presence of Bifidobacteria using sequence ID No. 4, sequence ID No. 5, sequence ID No. 6, sequence ID No. 7, sequence ID No. 8 or sequence ID No. 9 or a derivative, fragment or mutant thereof.

24 : A method of screening for the presence of Bifidobacteria using a two-component signal transduction system comprising a gene encoding sequence ID No. 4 and a gene sequence encoding sequence ID No. 5 or a derivative, fragment or mutant thereof.

25: A method of screening for the presence of Bifidobacteria using a two-component signal transduction system comprising a gene encoding sequence ID No. 6 and a gene sequence encoding sequence ID No. 7 or a derivative, fragment or mutant thereof.

26: A method of screening for the presence of Bifidobacteria using a two-component signal transduction system comprising a gene encoding sequence ID No. 8 and a gene sequence encoding sequence ID No. 9 or a derivative, fragment or mutant thereof.

27: A method as claimed in claim 22 wherein the Bifidobacteria is Bifidobacterium infantis UCC35624.

28: Use of a protein as claimed in claim 5 in the prophylaxis and/or treatment of undersirable inflammatory activity.

29: Use of a protein as claimed in claim 28 or an active derivative, fragment or mutant thereof in the prevention and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

30: Use of a protein as claimed in claim 29 or an active derivative, fragment or mutant thereof in the prophylaxis and/or treatment of undesirable gastrointestinal inflammatory activity such as; inflammatory bowel disease such as Crohns disease or ulcerative colitis; irritable bowel syndrome; pouchitis; or post infection colitis.

31: Use of a protein as claimed in claim 29 or an active derivative, fragment or mutant thereof in the prophylaxis and/or treatment of gastrointestinal cancer(s).

32: Use of a protein as claimed in claim 29 or an active derivative, fragment or mutant thereof in the prophylaxis and/or treatment of systemic disease such as rheumatoid arthritis.

33: Use of a protein as claimed in claim 29 or an active derivative, fragment or mutant thereof in the prophylaxis and/or treatment of autoimmune disorders due to undesirable inflammatory activity.

34: Use of a protein as claimed in claim 29 or an active derivative, fragment or mutant thereof in the prophylaxis and/or treatment of cancer due to undesirable inflammatory activity.

35: Use of a protein as claimed in claim 29 or an active derivative, fragment or mutant thereof in the prophylaxis of cancer.

36: Use of a protein as claimed in claim 29 or an active derivative, fragment or mutant thereof in the prophylaxis and/or treatment of diarrhoeal disease due to undesirable inflammatory activity, such as Clostridium difficile associated diarrhoea, Rotavirus associated diarrhoea or post infective diarrhoea or diarrhoeal disease due to an infectious agent, such as E. coli.

37: Use of a protein as claimed in claim 29 or an active derivative, fragment or mutant thereof in the preparation of anti-inflammatory biotherapeutic agents for the prophylaxis and/or treatment of undesirable inflammatory activity.
